(19) United States
(12) Patent Application Publication
(10) Pub. No.: US 2024/0355424 A1
(43) Pub. Date: Oct. 24, 2024

(54) CLINICAL DIAGNOSTICS USING GLYCANS

(71) Applicant: The Regents of the University of California, Oakland, CA (US)

(72) Inventors: Nathan E. Lewis, San Diego, CA (US); Wan Tien Chiang, San Diego, CA (US); Benjamin P. Kellman, San Diego, CA (US); Bokan Bao, San Diego, CA (US); Matthew Songmin Schinn, West Henrietta, NY (US)

(21) Appl. No.: 18/688,235

(22) PCT Filed: Aug. 31, 2022
(86) PCT No.: PCT/US2022/042156

§ 371 Date: Feb. 29, 2024

Related U.S. Application Data
(60) Provisional application No. 63/239,602, filed on Sep. 1, 2021.

(51) Int. Cl. G16B 40/10; G01N 30/02; G01N 30/72; G01N 30/88; G16H 50/20

(52) U.S. Cl. CPC G16B 40/10; G01N 30/7233; G01N 30/88; G16H 50/20; G01N 2030/027; G01N 2030/8813

(57) ABSTRACT

Method for using glycan or glycomics data for classification, diagnosis, prognosis, subject stratification, or therapy decision making based on a sample with glycans or glycosylated molecules.

### CROSS REFERENCE TO RELATED APPLICATIONS

This application claims the priority benefit of U.S. Provisional Application No. 63/239,602 filed Sep. 1, 2021, which is incorporated herein by reference.

### GOVERNMENT SPONSORSHIP

This invention was made with government support under GM119850 awarded by the National Institutes of Health. The government has certain rights in the invention.

### TECHNICAL FIELD

The present invention relates to a method of enhancing the use of glycans for diagnostics through the use of glycan substructure analysis and glycan sequencing technologies for clinical diagnostic, prognostic and therapeutic applications.

### BACKGROUND

Molecular changes are associated with many human diseases, which promise clinicians to perform diagnosis, evaluate therapeutic efficacy and predict disease recurrence.¹⁻⁶ Complex carbohydrates coat most cells, modify membrane lipids, impact the folding and function of most secreted and membrane proteins, and are critical components of the extracellular matrix.⁷ Unsurprisingly, alterations in glycosylation are associated with the pathophysiology of diverse diseases ranging from Alzheimer's to cancer.⁸⁻¹⁰ Several hallmarks of glycosylation alterations in cancer have been successfully discovered in the past decades, including: global sialylation increased, fucosylation increased, increased branching and bisecting GlcNAc N-glycans, and truncated O-glycans.¹¹ While the discovered aberrant glycosylation provides great promise for diagnosis and treatment of cancer,¹² the potential of glycans as disease markers has largely gone unrealized due to several major grand challenges.^{13,14}

The first grand challenge is the current state-of-the-art glycan measurement techniques involve difficult protocols including glycan release and purification, enzymatic digestions, and/or mass spectrometry, and these require extensive hands-on time, experimental resources, and experience to deploy.¹⁵ Mass spectrometry (MS) and lectin microarray are two major glycan analytical approaches, which have been developed for decades and successfully applied in much glycomics research.^{16,17} However, these two methods have their own limitations.^{18,19} Specifically, lectin microarrays are a high throughput technology, but they can't provide precise information about structure, linkage, and position of glycans. On the other hand, while MS-based methods can reliably identify the structure, linkage, and position of glycans, these methods are a lower-throughput technology due to their time-consuming and laborious procedures. Thus, there is a critical need for technologies that can sequence complex glycans with the ease currently enjoyed by nucleic acids. Thus, there is a critical need for technologies that can sequence complex glycans with the ease currently enjoyed by nucleic acids.²⁰

Another grand challenge is the rapid and accurate comparison of glycoprofiles remains largely difficult with the size, sparsity, heterogeneity and interdependence within such datasets.²¹ A glycoprofile provides glycan structure and abundance information, and each glycan is usually treated as an independent entity. Furthermore, in any one glycoprofile, only a tiny percentage of all possible glycans may be detected. Thus, if there is a significant perturbation to glycosylation in a dataset, only a few glycans, if any, may overlap between samples. However, these non-overlapping glycans may only differ in their synthesis by as few as one enzymatic step, requiring deliberate manual coding. Each glycan changing abundance in glycomics data influences the rest, thus confounding statistical analyses. This becomes difficult and laborious for large scale glycomics datasets needed to train sophisticated statistical models. A recent integrative analysis of lectin microarray and mass spectrometry analysis reveals altered N-linked glycosylation of hepatitis C proteins.²² This study demonstrated that, while lectins can identify altered glycan features of the glycan terminals, the detailed glycan structures identified by mass spectrometry remain largely unknown from the lectin profiles.

To address the grand challenges of glycan analytical method, the inventors recently developed a method for determining glycan structures and quantifying glycans using lectin profiling.²³ It was further shown that GlyCompare²¹ can be used for processing glycomics data to clarify analyses and greatly improve statistical power. Specifically, GlyCompare uses the biosynthetic history of each measured glycan to reveal abundances of shared biosynthetic intermediates.²¹ These substructure abundances clarify biological similarity between samples, and increased statistical power in glycomic data analysis. However, improved methods are needed for translating complex changes in glycans into clinical strategies.

### SUMMARY OF THE INVENTION

The present invention provides, in embodiments, methods for translating complex changes in glycans into clinical strategies, including an integrated method termed GLY-Seq Diagnostics (GSD) that enables a user to easily quantify a glycoform from a lectin profile and to effectively translate the quantified glycoprofile for informing clinical diagnostics, prognosis and therapeutic interventions. The invention includes the use of glycan substructure analysis for enhancing glycan-based diagnostics using a two-step process: 1) quantifying glyco-motif profiles and 2) applying GSD-classifier on biological samples to be classified (FIG. 1).

In this first step (FIG. 1), users measure glycosylation on a tissue, cell, biomolecule, or oligosaccharide. Glycans can be quantified by chromatography or mass spectrometry. In other applications, glycan structures can be determined by incubating the sample with more than one carbohydrate-binding molecule (e.g., lectin, Lectenz, antibody, nanobody, aptamer, etc.). The binding can be detected by any means known to one skilled in the art, such as fluorescence microscopy, immunohistochemistry, biotin-streptavidin, nucleotide sequencing, etc. detected using analysis by microscopy, flow or mass cytometry, sequencing, etc. The magnitude of binding is then transformed to a profile of possible glycan motifs recognized by the carbohydrate-binding molecule.

Then, the carbohydrate-binding molecule profile is used to determine glycan structure using machine learning approaches, trained from known glycoprofiles that map the carbohydrate binding molecule profile to the optimal glycoprofile. The invention then employs GlyCompare to leverage the biosynthetic history (glyco-motif profiles) of each glycan to reveal biosynthetic intermediate abundance from measured glycans. In this second step (FIG. 1B), the invention employs a classifier, trained from known control and disease classes, that classifies samples based on glyco-motif profiles. The invention enables glycan-based diagnostics, prognosis determination, therapeutic decision making and administration.

### BRIEF DESCRIPTION OF THE DRAWINGS

FIG. 1. A novel approach, GLY-Seq Diagnostics (GSD), for disease diagnostic, prognostic and therapeutic applications. The presented GSD-system comprises two steps. (STEP I) A schematic view of samples (e.g., cells or tissues presenting glycans), in which the glycans to be measured can be on samples (see details in Methods). Glycan motifs can be identified by binding carbohydrate-binding molecules, such as lectins. The resulting lectin binding patterns reveal the presence of key epitopes, and the original glycan species (MS-glycoprofile) is recapitulated from lectin binding patterns via a neural network. By applying GlyCompare²¹, the glycan substructure (glyco-motif profile) of each glycan can be derived for each sample. (STEP II) The glyco-motif profiles are subsequently used to train and interrogate an AI model that detects tumor-driven glycan signals and classifies the samples into different tumor classes.

FIGS. 2A-2C. Performance of glycoprofile reconstruction using neural networks. (FIG. 2A) A schematic view of the framework of the neural network-based method for predicting the mass-spectrometry-derived glycoprofiles: the lectin profile (input; red greyscale nodes), the predicted mass-spectrum glycoprofiles (output; blue greyscale nodes), and the neural network with two hidden layers and neurons (grey greyscale nodes). The boxplots of performance (R) for the glycoprofiles prediction from their corresponding lectin profiles using different neural network structures (number of layers and neurons). Each box represents the performance of 10 fold-cross validation of 100 random neural networks with the indicated topology. (FIG. 2B) The scatter plot of predicted glycan abundance versus experimental glycan abundance for the best performance neural network (1 hidden layer that contains 40 neurons). (FIG. 2C) Glycoprofiling of gastric cancer²⁴(17 samples; 3 normal mucosa: P8H, P9H and P10H; 6 gastric adenocarcinoma tumors: P1TS, P1TI, P2TS, P3TS, P4TS, P4TI; 8 normal mucosa of tumor-adjacent stomachs: P6TA-FS, P6TA-FG, P6TA-AS, P6TA-AG, P5TA-AS, P5TA-AG, P7TA-AS, P7TA-AG) of mucin O-glycosylation. The y-axis presents the relative abundances of indicated () glycans defined by their m/z value. The blue bars are the experimentally measured glycans, and the red bars are predicted by the best performance neural network described in (FIG. 2B). ‘R’ is Pearson correlation coefficient between the predictive glycans vs. the experimentally measured glycans.

FIG. 3. The importance of lectins for the best performing neural network. The importance of lectins used in the best performing neural network (FIG. 2B) for reconstructing the mass-spectrum glycoprofiles. The glycan epitopes specifically bound by the lectins are indicated on the top panel of the bar plot.

FIGS. 4A-4D. Performance of glycoprofile reconstruction with low-abundance glycans filtered at different levels. The scatter plot of predicted glycan abundance versus experimental glycan abundance for the glycoprofile reconstruction with lowly expressed glycans filtered at different levels. The glycoprofiles contain the glycans with their normalized glycan abundances greater than 1.0 (FIG. 4A), 0.7 (FIG. 4B), 0.5 (FIG. 4C) and 0.0 (FIG. 4D).

FIGS. 5A-5C. Glycoprofile reconstruction using neural networks with lowly expressed glycans filtered at different levels. The glycoprofiles of a gastric cancer dataset²⁴, profiling mucin O-glycosylation with lowly expressed glycans filtered. The glycoprofiles contain the glycans with their normalized glycan abundances greater than 0.7 (FIG. 5A), 0.5 (FIG. 5B) and 0.0 (FIG. C). The y-axis presents the relative abundances of indicated O-glycan m/z. The blue greyscale bars are the experimentally measured glycans, and the red greyscales bar are predicted by the best performing neural networks (FIGS. 6a-6c).

FIGS. 6a-6c. Increased power for identifying diagnostic markers shown through GlyCompare-based analysis of mucin-type O-glycans from normal, tumor-proximal, and gastrointestinal cancer biopsies, transformed to motif abundance. The differential analysis for the glycans and glyco-motifs derived from GlyCompare was conducted using the original glycoprofiles published by Jin et al.²⁴. (FIGS. 6a, 6b) Welch two-sample t-test P-value and false discovery rate (FDR) distributions for glycan abundance and glyco-motif abundance. (FIG. 6c) Multiple core 2 substructures were found to be depleted in gastrointestinal cancer relative to normal tissue. Not all linkages are specified, only those relevant to the substructure definition. The information of log fold changes (logFC) and the FDR are presented next to each substructure.

FIGS. 7A-7B. Differential analysis of the glycans and glyco-motifs (derived from GlyCompare) for the normal and gastrointestinal cancer biopsies. We conducted the differential analysis for the glycans and glyco-motifs derived from GlyCompare using the glycoprofiles reconstructed by the best performing neural network models (FIG. 2B). (FIG. 7A) Welch two-sample t-test P-value and false discovery rate (FDR) distributions for glycan abundance. No core 2 glycans were found to be statistically depleted in gastrointestinal cancer relative to normal tissue. (FIG. 7B) Welch two-sample t-test P-value and false discovery rate (FDR) distributions for glyco-motif abundance. Multiple core 2 substructures (IDs=L26789, L26814, L26950) were found depleted in gastrointestinal cancer relative to normal tissue. Not all linkages are specified, only those relevant to the substructure definition. The information of log fold changes (logFC) and the FDR are presented next to each substructure.

FIG. 8. Differential analysis of the glyco-motifs derived from GlyCompare. The differential analysis for all 10 glyco-motifs derived from GlyCompare using the glycoprofiles reconstructed by the best performing neural network models (FIG. 2B). The red greyscales rectangles around the graphs indicate the three substructures (IDs=L26789, L26814, L26950) depleted in gastrointestinal cancer relative to normal tissue.

FIGS. 9A-9E. Performance of GLY-Seq Diagnostics (GSD) classifier for gastric tumor stratification. (FIG. 9A) A schematic view of the framework of the neural network-based method for predicting the gastric tumor stratification: the glyco-motif profile (input; red greyscales nodes), the predicted gastric tumor class (output; blue greyscale nodes), and the neural network with two hidden layers and neurons (grey nodes). (FIGS. 9B-9E) The scatter plot of gastric tumor stratification for the glyco-motif profiles derived from the reconstructed glycoprofiles (FIGS. 6a-6c and 7A-7B). The glycoprofiles contain the glycans with their normalized glycan abundances greater than 1.0 (FIG. 9B), 0.7 (FIG. 9C), 0.5 (FIG. 9D) and 0.0 (FIG. 9E).

FIG. 10. The importance of glyco-motifs for the best performing neural network. The importance of glyco-motifs used in the best performing neural network models (FIG. 9B) for stratifying the gastric tumor samples. The glycan substructures are illustrated below the indicated glyco-motifs. The three red greyscales bars highlight the three glyco-motifs (L26789, L26950 and L26814) that are identified as significantly differential in the comparison between tumor and normal samples.

FIGS. 11a-11f. The core methodology for transforming glycoprofiles to glyco-motif profiles using GlyCompare. (FIG. 11a) and (FIG. 11b), A glycoprofile with structure and relative abundance annotation is obtained. The glycans are decomposed to a substructure set S, and the presence/absence of each substructure is recorded. Presence/absence vectors are weighted by the glycan abundance, and are summed into a substructure vector p. (FIG. 11c) Example glycoprofiles are transformed to substructure vectors as (FIG. 11a) and (FIG. 11b). (FIG. 11d) a substructure network is constructed to identify the non-redundant glyco-motifs that change in abundance from their precursor substructures. (FIG. 11e) The glycoprofiles can then be compared by their glyco-motif vectors to generate more meaningful clusters. Both glycoprofiles and substructures can be clustered for similarity analysis. (FIG. 11f) Core structure information can be visualized from a substructure cluster. For example, four substructures with different weights were aligned together, and the monosaccharides with a weight over 51% were preserved.

FIGS. 12A-12D. Analysis of site-specific N-glycosylation in brain. (FIG. 12A) Compositional site-specific N-glycan data from brain^{25.} (FIG. 12B) The same compositional data was substructure-decomposed to calculate substructure abundances presented in another biclustered heatmap. (FIG. 12C), (FIG. 12D) The Pearson correlation coefficient was calculated for the compositional and composition substructure abundance for each glycosylation site across proteins. The resulting correlation coefficients are presented as biclustered heatmaps. Biclustering used a complete agglomerative approach.

FIGS. 13A-13C. Performance of glycoprofile reconstruction using neural networks. (FIG. 13A) A schematic view of the framework of the neural network-based method for predicting the mass-spectrometry-derived glycoprofiles: the lectin profile (input; red greyscales nodes), the predicted mass-spectrometry glycoprofiles (output; blue greyscales nodes), and the neural network with two hidden layers and neurons (grey nodes). The boxplots of performance (R) for the glycoprofiles prediction from their corresponding lectin profiles using different neural network structures (number of layers and neurons). Each box represents the performance of 10 fold-cross validation of 100 random neural networks with the indicated topology. (FIG. 13B) The scatter plot of predicted glycan abundance versus experimental glycan abundance for the best performing neural network (3 hidden layer that contains 20 neurons). (FIG. 13C) Glycoprofiling of prostate cancer³⁷ (Table 3; 23 samples; 10 normal patients: A-I; 10 prostate cancer patients at T1c clinical tumor stage: K, L, N, O, P, Q, R, S, V, and W; 3 prostate cancer patients at T2 clinical tumor stage: M, T, and U) of PSA N-glycosylation. The y-axis presents the relative abundances of indicated N-glycans defined by their m/z value. The blue greyscales bars are the experimentally measured glycans, and the red greyscales bars are predicted by the best performing neural network described in (FIG. 13B). ‘R’ is Pearson correlation coefficient between the predictive glycans vs. the experimentally measured glycans.

FIG. 14. The importance of lectins for the best performing neural network. The importance of lectins used in the best performance neural network (FIG. 13B) for reconstructing the mass-spectrometry glycoprofiles. The glycan epitopes specifically bound by the lectins are indicated on the top panel of the bar plot.

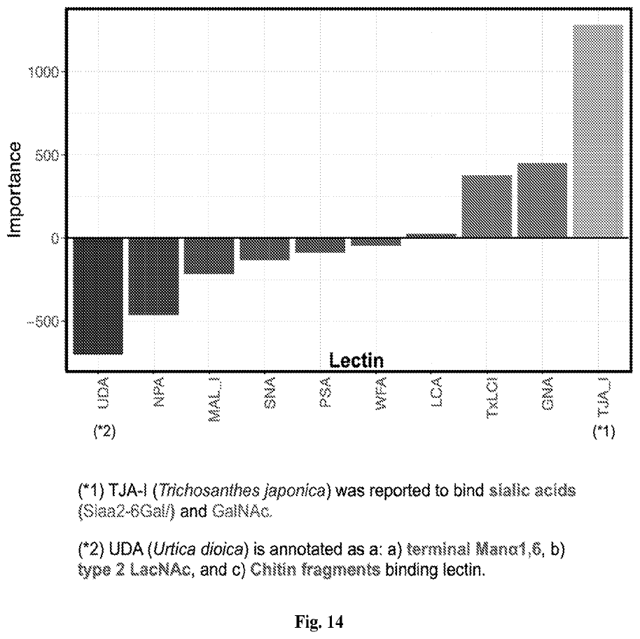

FIG. 15. Differential analysis of the glycans for the normal and prostate cancer (T1c and T2 tumor stages) biopsies. We conducted the differential analysis for the glycans using the experimentally measured glycoprofiles. (FIG. 15A) P-value distributions of ordinal regression test for glycan abundance. We find 1 glycan on PSA statistically enriched in T2 and T1c stage of prostate cancer relative to normal tissue. (FIG. 15B) Boxplots of differential abundance of glycans. The red rectangles indicate the only glycan (ID=G48) enriched on PSA in T2 and T1c stage of prostate cancer relative to normal tissue (T2>T1c>normal).

FIGS. 16A-16B. Differential analysis of the glyco-motifs derived from GlyCompare. We conducted the differential analysis (ordinal regression test) for all glyco-motifs derived from GlyCompare using the experimentally measured glycoprofiles. The red greyscales rectangles indicate the only differentially expressed substructures (IDs=L104793) enriched in prostate cancer relative to normal tissue (T2>T1c>normal).

FIGS. 17A-17B. Differential analysis of the glycans for the normal and prostate cancer (T1c and T2 tumor stages) biopsies. We conducted the differential analysis for the glycans using the glycoprofiles reconstructed by the best performing neural network models (FIG. 13B). (FIG. 17A) P-value distributions of ordinal regression test for glycan abundance. We find 2 glycans on PSA statistically depleted in T2 and T1c stage of prostate cancer relative to normal tissue. (FIG. 17B) Boxplots of differential abundance of glycans. The red greyscales rectangles indicate the two glycans (ID=G2 and G62) depleted on PSA in T2 and T1c stage of prostate cancer relative to normal tissue (T2<T1c<normal).

FIGS. 18A-18B. Differential analysis of the glyco-motifs derived from GlyCompare. We conducted the differential analysis (ordinal regression test) for all glyco-motifs derived from GlyCompare using the glycoprofiles reconstructed by the best performing neural network models (FIG. 13B). The red greyscales rectangles indicate the nine differentially expressed substructures: five (IDs=L144, L104491, L14134, L17102, and L30590) are depleted and four (IDs=L28, L46, L102, and L26664) are enriched in prostate cancer relative to normal tissue.

FIGS. 19A-19B. Performance of GLY-Seq Diagnostics (GSD) classifier for prostate tumor stratification based on BayesNet model #1. (FIG. 19A) The resampled data for testing the Bayesian network-based classifier. (FIG. 19B) The Bayesian network-based model #1 for predicting the prostate tumor stratification: the selected glyco-motifs as input and the predicted prostate tumor class (Normal, T1c, or T2). The right panel showed the conditional probability of indicated glyco-motif under different relative abundance range.

FIGS. 20A-20B. Performance of GLY-Seq Diagnostics (GSD) classifier for prostate tumor stratification based on BayesNet model #2. (FIG. 20A) The resampled data for testing the Bayesian network-based classifier. (FIG. 20B) The Bayesian network-based model #2 for predicting the prostate tumor stratification: the selected glyco-motifs as input and the predicted prostate tumor class (Normal, T1c, or T2). The right panel showed the conditional probability of indicated glyco-motif under different relative abundance range.

FIG. 21. A computer control system that is programmed or otherwise configured to implement methods provided herein.

### DETAILED DESCRIPTION

All publications, patents, and patent applications mentioned in this specification are herein incorporated by reference to the same extent as if each individual publication, patent, or patent application was specifically and individually indicated to be incorporated by reference.

In embodiments, the present invention provides a method of diagnosis, prognosis, or treatment of a subject for a disease or condition, the method comprising, providing a sample comprising glycans or glycosylated molecules from the subject; quantifying glyco-motif profiles in the sample, wherein substructures of glycans are used as features for classification; and translating the quantified glyco-motif profile for diagnosis, prognosis, or treatment.

In some embodiments, the glyco-motif profiles are quantified by measuring glycans using mass spectrometry or chromatography and generating a carbohydrate-binding molecule profile of glyco-motifs recognized by the carbohydrate-binding molecules.

In some embodiments, the glyco-motif profiles are quantified by incubating the sample with more than one carbohydrate-binding molecule, and generating a carbohydrate-binding molecule profile of glyco-motifs recognized by the carbohydrate-binding molecules.

In embodiments, the carbohydrate-binding molecule profile of glyco-motifs is used to determine glycan structure using a machine learning approach trained from known glycoprofiles that map the carbohydrate-binding molecule profile to an actual glycoprofile.

In embodiments, the glyco-motif profiles of each glycan are quantified to reveal biosynthetic intermediate abundance from measured glycans.

In embodiments, the translating step is performed by employing a classifier trained from known control and disease classes that classifies samples based on glyco-motif profiles for diagnosis, prognosis, or treatment.

In embodiments, substructures of glycans are used as features for classification using a method of machine learning, support vector machines, regression model, and/or neural networks.

In embodiments, the glyco-motif profiles are quantified by decomposing glycan measurements from mass spectrometry or chromatography, or reconstructed from carbohydrate binding molecule profiles.

In embodiments, the disease can be used for diagnosis, prognosis and treatment of many diseases (e.g., cancer, metabolic disease, immune diseases, reproductive health indications, etc.) or conditions, characterized by glycan abnormalities. In embodiments, the disease is gastric cancer or prostate cancer.

In embodiments, the methods of treatment comprise further administering to a subject in need thereof an effective amount of therapy to treat the disease or condition. In embodiments, the effective amount is determined by the level of disease or condition correlation, or stratification of disease or condition, determined through translation of the data.

In embodiments, the present invention provides a computer system, comprising:
one or more processors; and memory storing executable instructions that, as a result of execution, cause the one or more processors of the computer system to:
   a. quantify sample glycoprofiles by mass spectrometry or chromatography or by binding of carbohydrate-binding molecules to glycans in the sample;
   b. transform the quantified glycoprofiles to glyco-motif profiles based on previous training data between other related samples;
   c. classify the glyco-motif profiles into a disease class, wherein substructures of glycans are used as features for classification; and
   d. predict most likely disease classes based on previous training data between other related samples.

In embodiments of the system, the invention provides that the sample is a tissue, a cell, a biomolecule, or an oligosaccharide.

In embodiments of the system, the invention provides that the glycoprofiles are quantified by carbohydrate-binding molecules, reconstructed with trained algorithms based on previous training data between other samples, before transforming to glyco-motif profiles. In embodiments, the carbohydrate-binding molecules are natural or synthetic molecules that detect carbohydrate or carbohydrate containing compounds.

In embodiments of the system, the invention provides that the carbohydrate-binding molecules are selected from a lectin, an antibody, a nanobody, an aptamer, or an enzyme.

In embodiments of the system, the quantifying is conducted by fluorescence microscopy, immunohistochemistry, biotin-streptavidin, or sequencing of nucleic acid barcodes.

In embodiments of the system, the transforming of glycans from carbohydrate binding profiles is conducted using trained algorithm approaches from convex optimization and/or machine learning, trained from known glycoprofiles.

In embodiments of the system, the invention provides that the classifying is conducted using trained algorithm approaches from machine learning, support vector machine, regression model and/or neural networks trained from known glycoprofiles.

In embodiments of the system, the invention provides that the predicting is conducted using trained algorithm approaches from machine learning, support vector machine, regression model and/or neural networks trained from known glycoprofiles.

In embodiments of the system, the invention provides that the disease is cancer, metabolic disease, immune disease, or reproductive health condition. In embodiments of the system, the invention provides that the disease is gastric cancer or prostate cancer.

These and other embodiments and combinations of the embodiments will be apparent to one of ordinary skill in the art upon a review of the detailed description herein.

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a composition, and/or a method that “comprises” a list of elements (e.g., components, features, or steps) is not necessarily limited to only those elements (or components or steps), but may include other elements (or components or steps) not expressly listed or inherent to the composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim. When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “quantifying” “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

In an aspect, the disclosure provides in addition to a method of prognosing or diagnosing a disease or condition, a method of treating or preventing a disease or disorder in a subject in need thereof, further comprising administering an effective amount of a pharmaceutical composition for treatment of the identified disease or conditions. In some embodiments, the disease or disorder is a glycosylation-related disease or condition. In some embodiments, the glycosylation-related disease or condition comprises a cancer, metabolic disease, immune disease (including autoimmune disease), inflammatory condition, congenital disorders of glycosylation, or reproductive health condition. In some embodiments, the disease is cancer, and in embodiments is gastric cancer or prostate cancer.

Multiple diseases and conditions contemplated for diagnosis, prognosis and treatment using the present invention are known to be associated with glycosylation irregularities, as described in Reilly, et al., Glycosylation in health and disease. Nat. Rev. Nephrol. 15, 346-366; doi: 10.1038/s41581-019-0129-4 (2019), which is incorporated herein by reference.

A non-exhaustive list of cancer types and/or stages that may be identified using machine-learning models described herein include the following: Adrenocortical Carcinoma (TCGA-ACC); Bladder Urothelial Carcinoma (TCGA-BLCA); Brain Lower Grade Glioma (TCGA-LGG); Breast Invasive Carcinoma (TCGA-BRCA); Cervical Squamous Cell Carcinoma and Endocervical Adenocarcinoma (TCGA-CESC); Cholangiocarcinoma (TCGA-CHOL); Colon Adenocarcinoma (TCGA-COAD); Lymphoid Neoplasm Diffuse Large B-cell Lymphoma (TCGA-DLBC); Esophageal Carcinoma (TCGA-ESCA); Gastric Adenocarcinoma (TCGA-GA); Glioblastoma Multiforme (TCGA-GBM); Head and Neck Squamous Cell Carcinoma (TCGA-HNSC); Kidney Chromophobe (TCGA-KICH); Kidney Renal Clear Cell Carcinoma (TCGA-KIRC); Kidney Renal Papillary Cell Carcinoma (TCGA-KIRP); Liver Hepatocellular Carcinoma (TCGA-LIHC); Lung Adenocarcinoma (TCGA-LUAD); Lung Squamous Cell Carcinoma (TCGA-LUSC); Mesothelioma (TCGA-MESO); Ovarian Serous Cystadenocarcinoma (TCGA-OV); Pancreatic Adenocarcinoma (TCGA-PAAD); Pheochromocytoma and Paraganglioma (TCGA-PCPG); Prostate Adenocarcinoma (TCGA-PRAD); Rectum Adenocarcinoma (TCGA-READ); Sarcoma (TCGA-SARC); Skin Cutaneous Melanoma (TCGA-SKCM); Stomach Adenocarcinoma (TCGA-STAD); Testicular Germ Cell Tumors (TCGA-TGCT); Thyroid Carcinoma (TCGA-THCA); Thymoma (TCGA-THYM); Uterine Carcinosarcoma (TCGA-UCEC); Uterine Corpus Endometrial Carcinoma (TCGA-UCS); Uveal Melanoma (TCGA-UVM).

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, “administering” comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

An “effective amount” of a therapeutic administration will depend upon the degree of correlation between the glyco-motif profiles and the subject's glycosylation-related disease or condition, among other variables such as the age, health, and weight, which is within the skill or one of ordinary skill to determine.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used in certain contexts herein, glycan is used to refer to complete monosaccharide polymer; a glycan substructure refers to a complete or incomplete monosaccharide polymer observable within at least one measured glycan; a glyco-motif refers to an enriched functional glycan substructure for a dataset or biological process. Note that both glycan epitopes (typically terminal glycan substructures recognized by lectins) and glycan cores (biosynthetic glycan substructures common to select types (e.g. N- or O-glycosylation) or modes (e.g. complex or high-mannose) of biosynthesis) are glyco-motifs as they are biologically functional, interpretable and will be enriched in datasets selecting for specific glycan presentation of biosynthesis.

Glycoconjugates (e.g., a glycopeptide) as described herein may comprise one or more glycosylation features or glycans decorating a glycosite of an amino acid sequence. A glycosylation feature may comprise one or more monosaccharides linked glycosidically. A glycosylation feature may be present or otherwise associated with the glycosite. The association may comprise one or more covalent (e.g., glycosidic) bonds or the association may be non-covalent. A glycosylation feature may comprise any number of monosaccharides or derivatives. A glycosylation feature may comprise 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more monosaccharides or derivatives thereof.

Glycosylation features as described herein may comprise any monosaccharide or derivative thereof. Monosaccharides may comprise D-glucose (Glc), D-galactose (Gal), N-acetylglucosamine (GlcNAc), N-acetylgalactosamine (GalNAc), D-mannose (Man), N-acetylneuraminic acid (Neu5Ac), N-glycolylneuraminic acid (Neu5Gc), neuraminic acid (Neu), 2-keto-3-deoxynononic acid or 3-deoxy-D-glycero-D-galacto-nonulosonic acid (KDN), 3-deoxy-D-manno-2 octulopyranosylonic acid (Kdo), D-galacturonic acid (GalA), L-iduronic acid (IdoA), L-rhamnose (Rha), L-fucose (Fuc), D-xylose (Xyl), D-ribose (Rib), L-arabinofuranose (Araf), D-glucuronic acid (GlcA), D-allose (All), D-apiose (Api), D-fructofuranose (Fruf), ascarylose (Asc), and ribitol (Rbo). Derivatives of monosaccharides may comprise sugar alcohols, amino sugars, uronic acids, ulosonic acids, aldonic acids, aldaric acids, sulfosugars, or any combination or modification thereof. A sugar modification may comprise one or more of acetylation, propylation, formylation, phosphorylation, or sulfonation or addition of one or more of deacetylated N-acetyl (N), phosphoethanolamine (Pe), inositol (In), methyl (Me), N-acetyl (NAc), O-acetyl (Ac), phosphate (P), phosphocholine (Pc), pyruvate (Pyr), sulfate(S), sulfide (Sh), aminoethylphosphonate (Ep), deoxy (d), carboxylic acid (-oic), amine (-amine), amide (-amide), and ketone (-one). Such modifications may be present at any position on the sugar, as designated by standard sugar naming/notation. In some cases, a glycosytic addition of a monosaccharide to another monosaccharide is considered a polymerizing modification that gives rise to a glycans. In some embodiments, at least 1, 2, 3, 4, 5, 6, 7, 8, 9, 10 or more modifications are present on the monosaccharide. In some embodiments, no more than 10, 9, 8, 7, 6, 5, 4, 3, 2, 1, or fewer modifications are present on the monosaccharide. Monosaccharides may comprise any number of carbon atoms. Monosaccharides may comprise any stereoisomer, epimer, enantiomer, or anomer. In some embodiments, monosaccharides comprise 3, 4, 5, 6, 7, 8, 9, 10, 11, 12, 13, 14, 15, or more carbon atoms.

In some embodiments, a glycosylation feature comprises glyceraldehyle, threose, erythrose, lyxose, xylose (Xyl), arabinose, ribose, talose, galactose (Gal), idose, gulose, mannose (Man), glucose (Glc), altrose, allose, sedoheptulose, mannoheptulose, N-acetyl-galactosamine (Glc2NAc), glucuronic acid (GlcA), 3-O-sulfogalactose (Gal3S), N-acetylneuraminic acid (Neu5Ac), 2-keto-3-deoxynonic acid (Kdn), and any combination thereof.

A glycosylation feature may comprise one monosaccharide. A glycosylation feature may comprise a plurality of monosaccharides. In such cases, the monosaccharides may be connected in any configuration through any suitable glycosidic bond(s). Glycosidic bonds between monosaccharides in a polysaccharide glycosylation feature may be alpha or beta and connect any two carbon atoms between adjacent monosaccharide residues through an oxygen atom. In some embodiments, the glycosylation feature of glycan is an N-linked, O-linked, C-linked, or S-linked glycan. In some embodiments, more than one glycosylation feature is present on a single biomolecule. The more than one glycosylation features may all be linked in the same manner (e.g., N-linked, O-linked, C-linked, S-linked), or they may be independently N-linked, O-linked, C-linked, or S-linked. Glycosylation features may be branched, linear, or both. Glycosylation features may be biantennary, triantennary, tetra-antennary, or any combination thereof. In some embodiments, the glycosylation feature comprises a polysaccharide epitope. In some embodiments, the glycosylation feature comprises high-mannose. In some embodiments, the glycosylation feature comprises sialylation. In some embodiments, the glycosylation feature comprises fucosylation. In some embodiments, the glycosylation feature comprises hybrid, complex, core or distally fucosylated, terminally sialylated, terminally galactosylated, terminally GlcNAc-ylated, GlcNAc-bisected, or poly-sialylated, or a combination thereof.

A glycosylation feature may be described in relative terms. A glycosylation feature may be described as increased or decreased with respect to the amount of a given monosaccharide in the glycosylation feature relative to a reference glycosylation feature. For example, a glycosylation feature may be described as an increase or increased in sialylation or fucosylation if the glycosylation feature comprises more sialic acid or fucose residues, respectively, than a reference glycan. Alternatively or additionally, a glycosylation feature may be described as increased or decreased with respect to the configuration (e.g., branched, linear, biantennary, tri-antennary, tetra-antennary, penta-antennary) of the glycosylation feature relative to a reference glycosylation feature. For example, a glycosylation feature may be described as an increase or increased in branching if the glycosylation feature comprises more branches than a reference glycosylation feature. In some embodiments, a glycosylation feature may be described as increased or decreased in one or more of high-mannose, sialylation, fucosylation, hybrid, complexity, core or distally fucosylation, terminal sialylation, terminal galactosylation, terminal GlcNAc-ylation, GlcNAc-bisection, or poly-sialylation, or any other glycosylation feature.

### Trained Algorithms

Methods and systems as described herein may employ one or more trained algorithms. The trained algorithm(s) may process or operate on one or more datasets comprising information about biomolecules (e.g., biomolecular features), biochemical features (e.g., lectin binding), glycans and glycosylation features, or any combination thereof. In some embodiments, the datasets comprise structural or sequence information about biomolecules. In some embodiments, the datasets comprise one or more datasets of glycosylation features. The one or more datasets may be observed empirically, derived from computational studies, be derived from or contained in one or more databases, or any combination thereof.

The trained algorithm may comprise an unsupervised machine learning algorithm. The trained algorithm may comprise a supervised machine learning algorithm. The trained algorithm may comprise a semi-supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise a self-supervised machine learning algorithm.

In some embodiments, a machine learning algorithm (or software module) of a platform as described herein utilizes one or more neural networks. In some embodiments, a neural network is a type of computational system that can learn the relationships between an input dataset and a target dataset. A neural network may be a software representation of a human neural system (e.g. cognitive system), intended to capture “learning” and “generalization” abilities as used by a human. In some embodiments, the machine learning algorithm (or software module) comprises a neural network comprising a CNN. Non-limiting examples of structural components of embodiments of the machine learning software described herein include: CNNs, recurrent neural networks, dilated CNNs, fully-connected neural networks, deep generative models, recurrent neural networks (RNNs), RNNs using long short-term memory (LSTM) units, and Boltzmann machines.

In some embodiments, a neural network comprises a series of layers termed “neurons.” In some embodiments, a neural network comprises an input layer, to which data is presented; one or more internal, and/or “hidden”, layers; and an output layer. A neuron may be connected to neurons in other layers via connections that have weights, which are parameters that control the strength of the connection. The number of neurons in each layer may be related to the complexity of the problem to be solved. The minimum number of neurons required in a layer may be determined by the problem complexity, and the maximum number may be limited by the ability of the neural network to generalize. The input neurons may receive data being presented and then transmit that data to the first hidden layer through connections' weights, which are modified during training. The first hidden layer may process the data and transmit its result to the next layer through a second set of weighted connections. Each subsequent layer may “pool” the results from the previous layers into more complex relationships. In addition, whereas conventional software programs require writing specific instructions to perform a function, neural networks are programmed by training them with a known sample set and allowing them to modify themselves during (and after) training so as to provide a desired output such as an output value. After training, when a neural network is presented with new input data, it is configured to generalize what was “learned” during training and apply what was learned from training to the new previously unseen input data to generate an output associated with that input.

In some embodiments, the neural network comprises ANNs. ANN may be machine learning algorithms that may be trained to map an input dataset to an output dataset, where the ANN comprises an interconnected group of nodes organized into multiple layers of nodes. For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (such as a DNN) is an ANN comprising a plurality of hidden layers, e.g., two or more hidden layers. Each layer of the neural network may comprise a number of nodes (or “neurons”). A node receives input that comes either directly from the input data or the output of nodes in previous layers, and performs a specific operation, e.g., a summation operation. A connection from an input to a node is associated with a weight (or weighting factor). The node may sum up the products of all pairs of inputs and their associated weights. The weighted sum may be offset with a bias. The output of a node or neuron may be gated using a threshold or activation function. The activation function may be a linear or non-linear function. The activation function may be, for example, a rectified linear unit (ReLU) activation function, a Leaky ReLU activation function, or other function such as a saturating hyperbolic tangent, identity, binary step, logistic, arctan, softsign, parametric rectified linear unit, exponential linear unit, softplus, bent identity, softexponential, sinusoid, sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training dataset and a gradient descent or backward propagation method so that the output value(s) that the ANN computes are consistent with the examples included in the training dataset.

The number of nodes used in the input layer of the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In some instances, the number of node used in the input layer may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or less. In some instances, the total number of layers used in the ANN or DNN (including input and output layers) may be at least about 3, 4, 5, 10, 15, 20, or greater. In some instances, the total number of layers may be at most about 20, 15, 10, 5, 4, 3, or less.

In some instances, the total number of learnable or trainable parameters, e.g., weighting factors, biases, or threshold values, used in the ANN or DNN may be at least about 10, 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, or greater. In some instances, the number of learnable parameters may be at most about 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 50, 10, or less.

In some embodiments of a machine learning software module as described herein, a machine learning software module comprises a neural network such as a deep CNN. In some embodiments in which a CNN is used, the network is constructed with any number of convolutional layers, dilated layers or fully-connected layers. In some embodiments, the number of convolutional layers is between 1-10 and the dilated layers between 0-10. The total number of convolutional layers (including input and output layers) may be at least about 1, 2, 3, 4, 5, 10, 15, 20, or greater, and the total number of dilated layers may be at least about 1, 2, 3, 4, 5, 10, 15, 20, or greater. The total number of convolutional layers may be at most about 20, 15, 10, 5, 4, 3, or less, and the total number of dilated layers may be at most about 20, 15, 10, 5, 4, 3, or less. In some embodiments, the number of convolutional layers is between 1-10 and the fully-connected layers between 0-10. The total number of convolutional layers (including input and output layers) may be at least about 1, 2, 3, 4, 5, 10, 15, 20, or greater, and the total number of fully-connected layers may be at least about 1, 2, 3, 4, 5, 10, 15, 20, or greater. The total number of convolutional layers may be at most about 20, 15, 10, 5, 4, 3, 2, 1, or less, and the total number of fully-connected layers may be at most about 20, 15, 10, 5, 4, 3, 2, 1, or less.

In some embodiments, the input data for training of the ANN may comprise a variety of input values depending whether the machine learning algorithm is used for processing sequence or structural data. In general, the ANN or deep learning algorithm may be trained using one or more training datasets comprising the same or different sets of input and paired output data.

In some embodiments, a machine learning software module comprises a neural network comprising a CNN, RNN, dilated CNN, fully-connected neural networks, deep generative models and deep restricted Boltzmann machines.

In some embodiments, a machine learning algorithm comprises CNNs. The CNN may be deep and feedforward ANNs. The CNN may be applicable to analyzing visual imagery. The CNN may comprise an input, an output layer, and multiple hidden layers. The hidden layers of a CNN may comprise convolutional layers, pooling layers, fully-connected layers and normalization layers. The layers may be organized in 3 dimensions: width, height and depth.

The convolutional layers may apply a convolution operation to the input and pass results of the convolution operation to the next layer. For processing images, the convolution operation may reduce the number of free parameters, allowing the network to be deeper with fewer parameters. In neural networks, each neuron may receive input from some number of locations in the previous layer. In a convolutional layer, neurons may receive input from only a restricted subarea of the previous layer. The convolutional layer's parameters may comprise a set of learnable filters (or kernels). The learnable filters may have a small receptive field and extend through the full depth of the input volume. During the forward pass, each filter may be convolved across the width and height of the input volume, compute the dot product between the entries of the filter and the input, and produce a two-dimensional activation map of that filter. As a result, the network may learn filters that activate when it detects some specific type of feature at some spatial position in the input.

In some embodiments, the pooling layers comprise global pooling layers. The global pooling layers may combine the outputs of neuron clusters at one layer into a single neuron in the next layer. For example, max pooling layers may use the maximum value from each of a cluster of neurons in the prior layer; and average pooling layers may use the average value from each of a cluster of neurons at the prior layer.

In some embodiments, the fully-connected layers connect every neuron in one layer to every neuron in another layer. In neural networks, each neuron may receive input from some number locations in the previous layer. In a fully-connected layer, each neuron may receive input from every element of the previous layer.

In some embodiments, the normalization layer is a batch normalization layer. The batch normalization layer may improve the performance and stability of neural networks. The batch normalization layer may provide any layer in a neural network with inputs that are zero mean/unit variance. The advantages of using batch normalization layer may include faster trained networks, higher learning rates, easier to initialize weights, more activation functions viable, and simpler process of creating deep networks.

In some embodiments, a machine learning software module comprises a recurrent neural network software module. A recurrent neural network software module may be configured to receive sequential data as an input, such as consecutive data inputs, and the recurrent neural network software module updates an internal state at every time step. A recurrent neural network can use internal state (memory) to process sequences of inputs. The recurrent neural network may be applicable to tasks such as handwriting recognition or speech recognition. The recurrent neural network may also be applicable to next word prediction, music composition, image captioning, time series anomaly detection, machine translation, scene labeling, and stock market prediction. A recurrent neural network may comprise fully recurrent neural network, independently recurrent neural network, Elman networks, Jordan networks, Echo state, neural history compressor, long short-term memory, gated recurrent unit, multiple timescales model, neural Turing machines, differentiable neural computer, and neural network pushdown automata.

In some embodiments, a machine learning software module comprises a supervised or unsupervised learning method such as, for example, support vector machines (“SVMs”), random forests, clustering algorithm (or software module), gradient boosting, logistic regression, and/or decision trees. The supervised learning algorithms may be algorithms that rely on the use of a set of labeled, paired training data examples to infer the relationship between an input data and output data. The unsupervised learning algorithms may be algorithms used to draw inferences from training datasets to the output data. The unsupervised learning algorithm may comprise cluster analysis, which may be used for exploratory data analysis to find hidden patterns or groupings in process data. One example of unsupervised learning method may comprise principal component analysis. The principal component analysis may comprise reducing the dimensionality of one or more variables. The dimensionality of a given variable may be at least 1, 5, 10, 50, 100, 200, 300, 400, 500, 600, 700, 800, 900, 1,000, 1,100, 1,200, 1,300, 1,400, 1,500, 1,600, 1,700, 1,800, or greater. The dimensionality of a given variables may be at most 1,800, 1,700, 1,600, 1,500, 1,400, 1,300, 1,200, 1,100, 1,000, 900, 800, 700, 600, 500, 400, 300, 200, 100, 50, 10, or less.

In some embodiments, the machine learning algorithm may comprise reinforcement learning algorithms. The reinforcement learning algorithm may be used for optimizing Markov decision processes (i.e., mathematical models used for studying a wide range of optimization problems where future behavior cannot be accurately predicted from past behavior alone, but rather also depends on random chance or probability). One example of reinforcement learning may be Q-learning. Reinforcement learning algorithms may differ from supervised learning algorithms in that correct training data input/output pairs are never presented, nor are sub-optimal actions explicitly corrected. The reinforcement learning algorithms may be implemented with a focus on real-time performance through finding a balance between exploration of possible outcomes (e.g., correct compound identification) based on updated input data and exploitation of past training.

In some embodiments, training data resides in a cloud-based database that is accessible from local and/or remote computer systems on which the machine learning-based sensor signal processing algorithms are running. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site.

The trained algorithm may accept a plurality of input variables and produce one or more output variables based on the plurality of input variables. The input variables may comprise one or more datasets indicative of a glycosylation feature. For example, the input variables may comprise a carbohydrate binding protein pattern, glycan structures, glycan features, clinical outcomes, diagnosis, or any combination thereof.

The trained algorithm may be trained with a plurality of independent training samples. Each of the independent training samples may comprise a carbohydrate binding protein pattern and glycan structures or glycan features and diagnosis. The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, at least about 500, at least about 1,000, at least about 1,500, at least about 2,000, at least about 2,500, at least about 3,000, at least about 3,500, at least about 4,000, at least about 4,500, at least about 5,000, at least about, 5,500, at least about 6,000, at least about 6,500, at least about 7,000, at least about 7,500, at least about 8,000, at least about 8,500, at least about 9,000, at least about 9,500, at least about 10,000, or more independent training samples.

The trained algorithm may be adjusted or tuned to improve one or more of the performance, accuracy, PPV, NPV, sensitivity, specificity, or AUC of associating the glycosylation feature. The trained algorithm may be adjusted or tuned by adjusting parameters of the trained algorithm (e.g., a set of cutoff values used to associate a glycosylation feature as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality predictions. For example, a subset of the data may be identified as most influential or most important to be included for making high-quality associations of carbohydrate binding protein patterns and glycan features or glycan features and diagnosis. The data or a subset thereof may be ranked based on classification metrics indicative of each parameter's influence or importance toward making high-quality associations. Such metrics may be used to reduce, in some embodiments significantly, the number of input variables (e.g., predictor variables) that may be used to train the trained algorithm to a desired performance level (e.g., based on a desired minimum accuracy, PPV, NPV, sensitivity, specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best association metrics.

Systems and methods as described herein may use more than one trained algorithm to determine an output (e.g., association of a carbohydrate binding protein patterns and glycan features or glycan features and diagnosis). Systems and methods may comprise 1, 2, 3, 4, 5, 6, 7, 8, 9, 10, 11, 12, 13, 14, 15, 16, 17, 18, 19, 20, or more trained algorithms. A trained algorithm of the plurality of trained algorithms may be trained on a particular type of data (e.g., carbohydrate binding protein patterns, glycan features, diagnosis, etc.). Alternatively, a trained algorithm may be trained on more than one type of data. The inputs of one trained algorithm may comprise the outputs of one or more other trained algorithms. Additionally, a trained algorithm may receive as its input the output of one or more trained algorithms.

The glycosylation feature may comprise one or more monosaccharides. The glycosylation feature may comprise mannose, sialic acid, fucose, GlcNAc, GalNAc, or any other monosaccharide, and combinations thereof. The glycosylation feature may comprise a polysaccharide epitope. In some embodiments, the glycosylation feature is an increase or decrease in a high-mannose in one of the variant sequences as compared to the reference sequence. In some embodiments, the glycosylation feature is an increase or decrease in a sialylation in one of the variant sequences as compared to the reference sequence. In some embodiments, the glycosylation feature is an increase or decrease in a high-mannose in one of the variant sequences as compared to the reference sequence. In some embodiments, the glycosylation feature is an increase or decrease in a glycosylation feature, such as monosaccharides or a glycan epitope or substructure.

In some embodiments, the likelihood may be expressed as a probability. In some embodiments, the likelihood may be expressed as a pseudo-probability. In some embodiments, the likelihood may be expressed as a ratio or product of one or more probabilities or pseudo-probabilities. In some embodiments, the likelihood may be expressed as a sum or difference of one or more probabilities. In some embodiments, the likelihood may be expressed as an odds ratio. In some embodiments, the likelihood may be expressed as the logarithm of an odds ratio.

In some embodiments, the method comprises diagnosing a disease or deciding on a therapeutic administration or therapy based at least in part on determining the glyco-motif pattern on a biological sample obtained from the patient.

Further provided herein are computer systems comprising a digital processing device comprising at least one processor, an operating system configured to perform executable instructions, a memory, and a computer program including instructions executable by the digital processing device to create an application for conducting the methods described herein.

Further provided herein are non-transitory computer-readable mediums comprising machine-executable code that, upon execution by one or more computer processors, implements a method for conducting the methods herein.

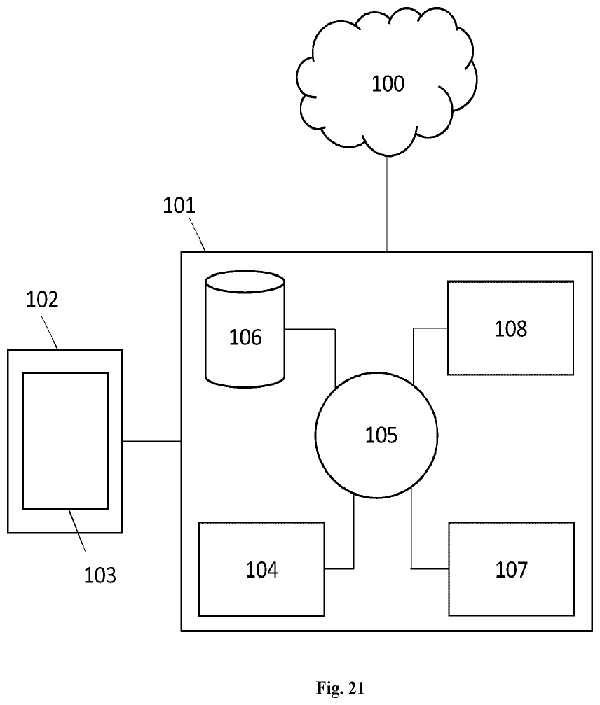

The present disclosure provides computer systems that are programmed to implement methods of the disclosure. FIG. 21 shows a computer system 101 that is programmed or otherwise configured to conduct the methods described herein.

The computer system 101 can regulate various aspects of analysis, calculation, and generation of the present disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 104 (e.g., random-access memory, read-only memory, flash memory), electronic storage unit 106 (e.g., hard disk), communication interface 108 (e.g., network adapter) for communicating with one or more other systems, and peripheral devices 107, such as cache, other memory, data storage and/or electronic display adapters. The memory 104, storage unit 106, interface 108 and peripheral devices 107 are in communication with the CPU 105 through a communication bus (solid lines), such as a motherboard. The storage unit 106 can be a data storage unit (or data repository) for storing data. The computer system 101 can be operatively coupled to a computer network (“network”) 100 with the aid of the communication interface 108. The network 100 can be the Internet, an internet and/or extranet, or an intranet and/or extranet that is in communication with the Internet.

In some embodiments, the network 100 is a telecommunication and/or data network. The network 100 can include one or more computer servers, which can enable distributed computing, such as cloud computing. For example, one or more computer servers may enable cloud computing over the network 100 (“the cloud”) to perform various aspects of analysis, calculation, and generation of the present disclosure. Such cloud computing may be provided by cloud computing platforms such as, for example, Amazon Web Services (AWS), Microsoft Azure, Google Cloud Platform, and IBM cloud. In some embodiments, the network 100, with the aid of the computer system 101, can implement a peer-to-peer network, which may enable devices coupled to the computer system 101 to behave as a client or a server.

The CPU 105 may comprise one or more computer processors and/or one or more graphics processing units (GPUs). The CPU 105 can execute a sequence of machine-readable instructions, which can be embodied in a program or software. The instructions may be stored in a memory location, such as the memory 104. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some embodiments, the circuit is an application specific integrated circuit (ASIC).

The storage unit 106 can store files, such as drivers, libraries and saved programs. The storage unit 106 can store user data, e.g., user preferences and user programs. In some embodiments, the computer system 101 can include one or more additional data storage units that are external to the computer system 101, such as located on a remote server that is in communication with the computer system 101 through an intranet or the Internet.

The computer system 101 can communicate with one or more remote computer systems through the network 100. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 101 via the network 100.

Methods as described herein can be implemented by way of machine (e.g., computer processor) executable code stored on an electronic storage location of the computer system 101, such as, for example, on the memory 104 or electronic storage unit 106. The machine executable or machine readable code can be provided in the form of software. During use, the code can be executed by the processor 105. In some embodiments, the code can be retrieved from the storage unit 106 and stored on the memory 104 for ready access by the processor 105. In some situations, the electronic storage unit 106 can be precluded, and machine-executable instructions are stored on memory 104.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Embodiments of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 102 that comprises a user interface (UI) 103 for conducting the methods described herein. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 105.

### EXAMPLES

### Example 1—Mucin-Type O-Glycosylation for Diagnosis of Gastric Cancer

Reconstructing the Mass-Spectrometry-Based Glycoprofiles from Lectin Profiles with a Neural Network

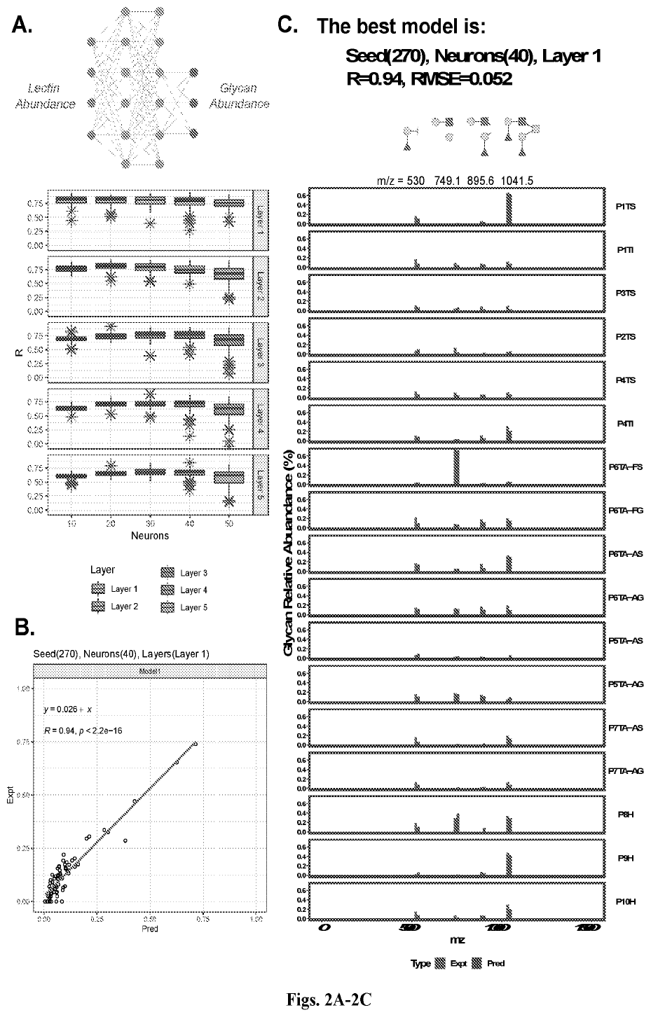

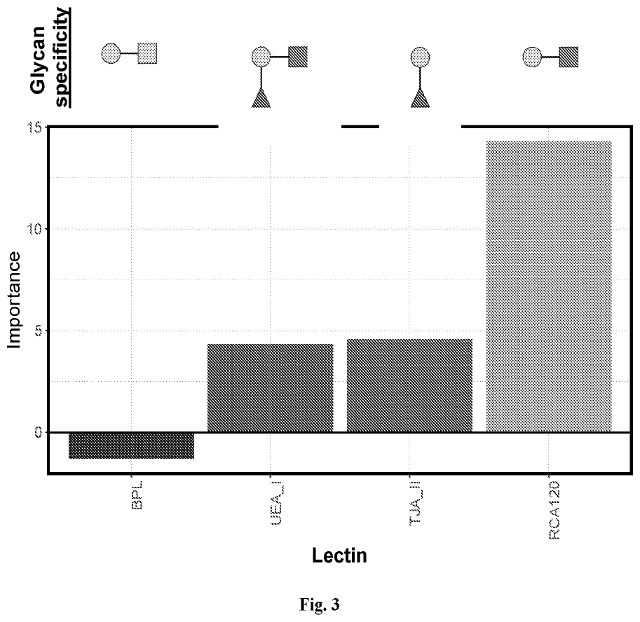

We have developed an effective method²³ for providing accurate prediction of the mass-spectrometry-based glycoprofiles from lectin profiles by learning a computational neural network model from the organism and glycosylation class of interest. Specifically, the neural network model can take any lectin profile and make predictions on its corresponding glycoprofile. Here we trained a neural network model on gastric glycomics data²⁴ (see details in Methods). To determine the optimal neural network topology, we assessed the performance using different combinations of hidden layer size and neuron size in each layer. Based on the ten-fold cross-validation, our results show that the neural network with 1 hidden layer (40 neurons) has the best average prediction power, in which the best model has excellent performance (Pearson correlation coefficient (R)=0.94, p<2.2e-16) (FIGS. 2A-2C). To further understand the importance of input lectins in neural networks, the relative importance of each lectin is quantified as the sum of the product of raw input-hidden and hidden-output connection weights between each input and output neuron and sums the product across all hidden neurons.^{26,27} Our results suggest that four lectins: RCA120 that specifically bound to terminal galactose (terminal Gal), TJA-II (terminal Sialylation), UEA-I (H-type 2 antigen), and BPL (Lewis x antigen) are essential for accurately reconstructing the gastric cancer glycoprofiles (FIG. 3). While these four lectins should be critically designated as probes to detect glycans in the GLY-Sequencing detection device (e.g., Microfluidic platforms, FACS, etc.) for the gastric tumor glycans profiled here, trial runs on all lectins can be used to identify the most important lectins for profiling the glycan patterns in the other disease of interest.

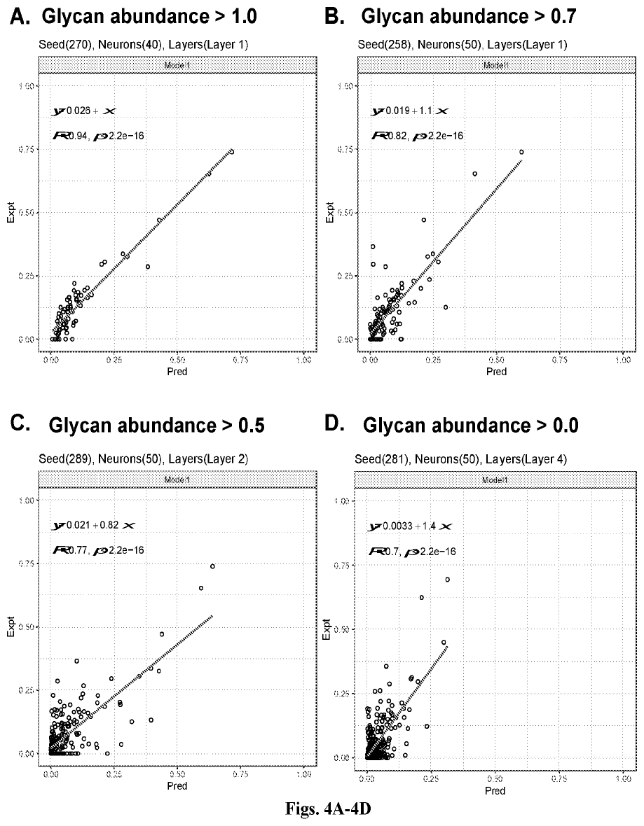

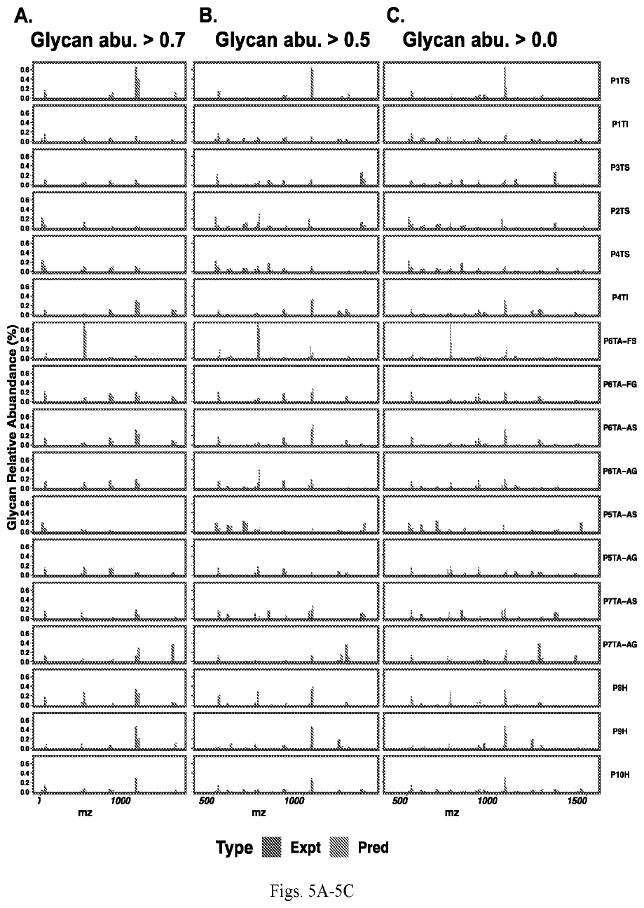

We further tested the robustness of model in reconstructing the mass-spectrometry-based glycoprofiles by filtering lowly expressed glycans at different levels. Our results show the performance of glycoprofile reconstruction with different levels of lowly expressed glycans filtered (R=0.94, 0.82, 0.77 and 0.7 at levels of 1.0, 0.7, 0.5 and 0.0, respectively) (FIGS. 4A-4D and 5A-5C). We found that our neural network models continued to predict highly accurate glycoprofiles (R>0.7) even with the noisy, lowly expressed glycans recruited. These results thus provide us confidence to apply the neural network models for robustly reconstructing the mass-spectrometry-based glycoprofiles from different levels of noise of lectin profiles.

### GlyCompare Reveals Tumor-Depleted Mucin-Type O-Glycans by Increasing the Statistical Power of Glycomics Data

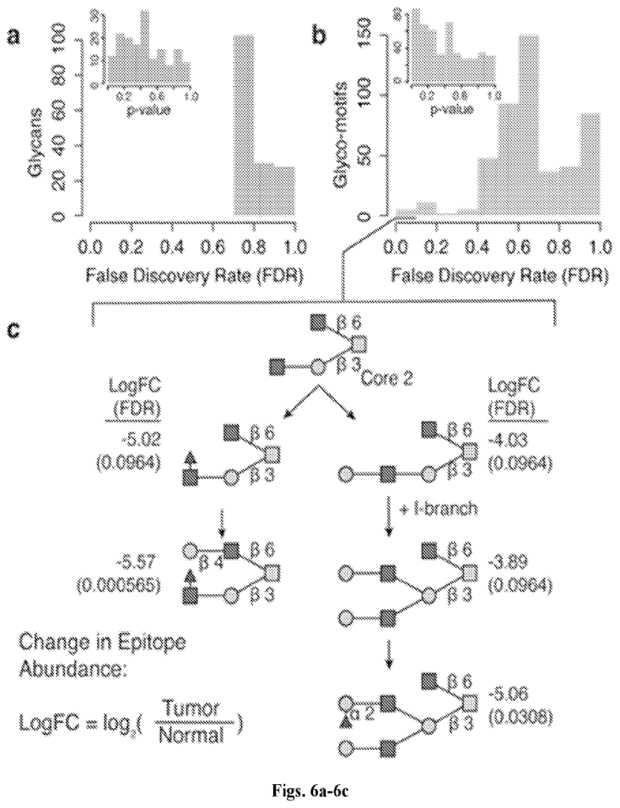

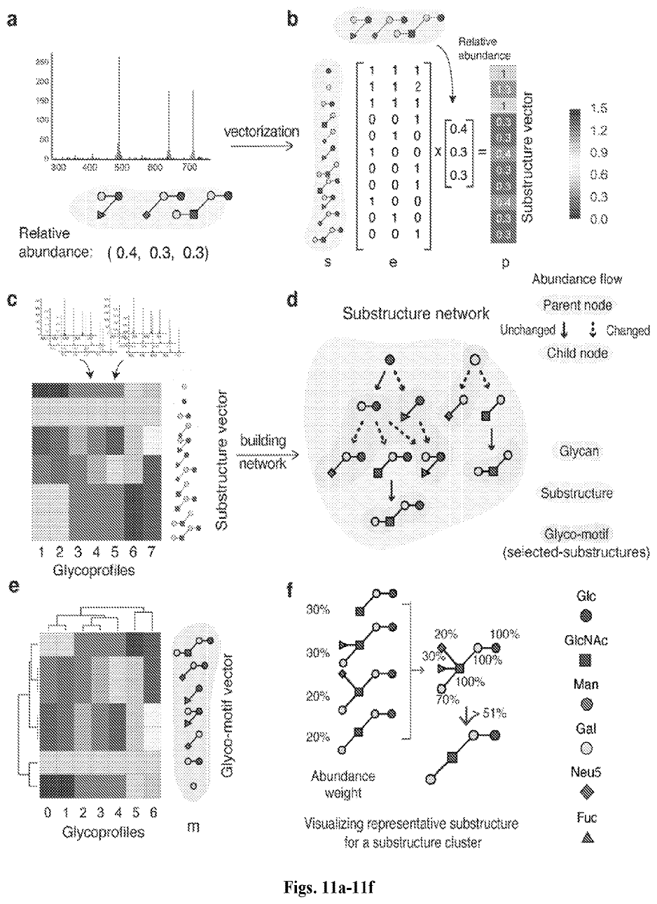

We demonstrated that the major obstacles of automatically identifying glycan structure from high throughput glycomics data lie in the limited sample size and the sparse shared glycans between cancer and normal samples, leading to the substantially decreased statistical power of statistics-based methods. To investigate whether we can computationally identify glyco-substructure signatures from glycomics data for stratifying subjects between normal and cancer, we started by using our method-GlyCompare (FIGS. 11a-11f) to calculate substructure abundance for mucin type O-glycans²⁴ (FIGS. 6a-6c). In the examination of the mucin-type O-glycans from tumor and normal samples, glycan abundance and motif abundance were compared (FIGS. 6a, 6b). We found that zero whole-glycan structures significantly distinguished between tumor and normal following multiple-test correction (FDR<0.1, FIG. 6a). Yet, after substructure decomposition using GlyCompare, we found five significantly depleted (FDR<0.1) mucin-type glycan motifs in gastrointestinal cancer²⁴ (FIG. 6b). We found a substantial depletion in the tumor samples of five core 2 structures. These structures included three fucosylated and two with I-branches. The largest structures were over 30-fold depleted in tumors (FDR<0.03, FIG. 6c). The core 2 depletion was noted as a nonsignificant trend in the original publication; we identified the specific core 2-type substructure depleted in tumors using substructure decomposition. Through this we demonstrate the increase in statistical power when using substructures. In addition, a later study also found significant depletion of multiple bi-GlcNAc core 2 and I-branched structures²⁴. Also consistent with the decrease in bi-GlcNAc core-2 structures in gastric cancer, low expression of B3GNT3 in stomach cancer is significantly associated with decreased overall survival²⁸. B3GNT3 is necessary for adding the second GlcNac to core 2 structure²⁹ and therefore upstream of all significantly depleted structures (FIGS. 6a-6c); B3GNT3 depletion could explain the observed differential glycosylation. The observation of significantly distinct substructures suggests GlyCompare provided increased statistical power to detect these distinguishing condition-enriched structures, and further showed continuity across similar structures was not evident in the original study.

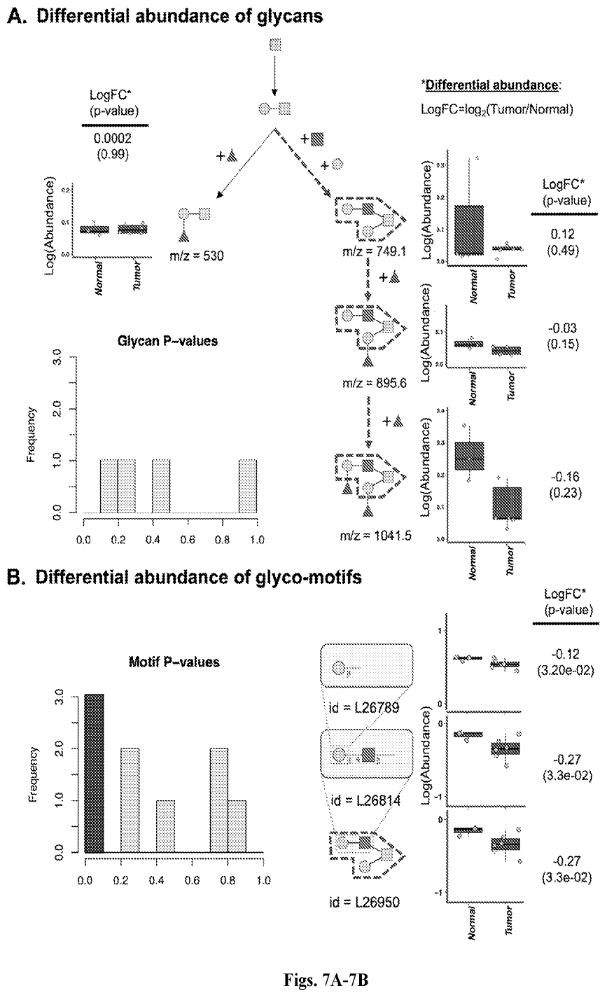

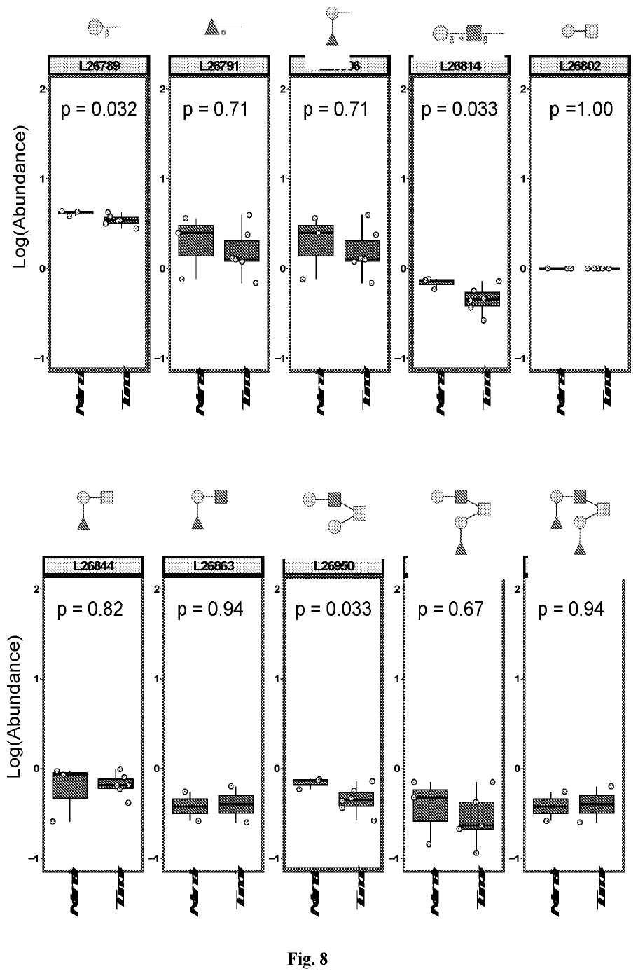

Next, we tested whether we could identify glyco-substructure signatures from the reconstructed glycomics data (FIGS. 2A-2C) using the best neural network model. In the re-examination of the mucin-type O-glycans from tumor and normal samples, we found zero whole-glycan structures significantly distinguished between tumor and normal following multiple-test correction (p value<0.05; FIG. 7A and Table 1). Again, after substructure decomposition using GlyCompare, we found three significantly depleted (p value<0.05) mucin-type glycan motifs (IDs=L26789, L26814, and L268950) in gastrointestinal cancer²⁴ (FIGS. 7B and 8; Table 2). Comparing the glycan structures and the glyco-motif structures, the identified glycan motif (L268950) clearly showed the altered biosynthetic intermediate abundances originated from the biosynthetic history of three core 2 structures (m/z=749.1, 895.6 and 1041.5; highlighted by the dashed polygons). The other two glyco-motifs (L26789 and L26814) are substructures of the glycan motif (L268950). Interestingly, the glycan motif (L268950) is the substructure of the fucosylated core 2 structure identified by GlyCompare to be substructure depleted in tumors using the experimental glycomics data (FIG. 6c). All these results demonstrated that GlyCompare can process the reconstructed glycomics data to clarify analyses and greatly improve statistical power.

TABLE 1
Glycan level statistics

| | Mean | Mean | Mean | | | |
|---|---|---|---|---|---|---|
| m/z | log(Abundance) | log(Tumor) | log(Normal) | statistic | p. value | logFC |
| 530 | 0.00 | 0.08 | 0.08 | 0.01 | 9.92E−01 | 0.00 |
| 749.1 | −0.08 | 0.04 | 0.12 | −0.83 | 4.93E−01 | −0.12 |
| 895.6 | −0.02 | 0.04 | 0.06 | −1.86 | 1.50E−01 | −0.03 |
| 1041.5 | −0.11 | 0.15 | 0.26 | −1.30 | 2.35E−01 | −0.16 |

TABLE 2
Glyco-motif level statistics

| | Mean | Mean | Mean | | | |
|---|---|---|---|---|---|---|
| Motif | log(Abundance) | (Tumor) | (Normal) | statistic | p. value | logFC |
| L26789 | −0.08 | 0.54 | 0.62 | −2.67 | 3.20E−02 | −0.12 |
| L26814 | −0.19 | −0.35 | −0.16 | −2.65 | 3.31E−02 | −0.27 |
| L26950 | −0.19 | −0.35 | −0.16 | −2.65 | 3.31E−02 | −0.27 |
| L26863 | −0.42 | −1.06 | −0.65 | −1.23 | 2.69E−01 | −0.60 |
| L27517 | −0.42 | −1.06 | −0.65 | −1.23 | 2.69E−01 | −0.60 |
| L27136 | −0.21 | −0.65 | −0.44 | −0.82 | 4.56E−01 | −0.30 |
| L26791 | −0.10 | 0.18 | 0.28 | −0.41 | 7.06E−01 | −0.14 |
| L26806 | −0.10 | 0.18 | 0.28 | −0.41 | 7.06E−01 | −0.14 |
| L26844 | 0.05 | −0.18 | −0.23 | 0.26 | 8.17E−01 | 0.07 |
| L26802 | 0.00 | 0.00 | 0.00 | 0.00 | 1.00 | 0.00 |

The GLY-Seq Diagnostics (GSD) Classifier can Successfully Classify Glyco-Motif Profile into Different Tumor Class

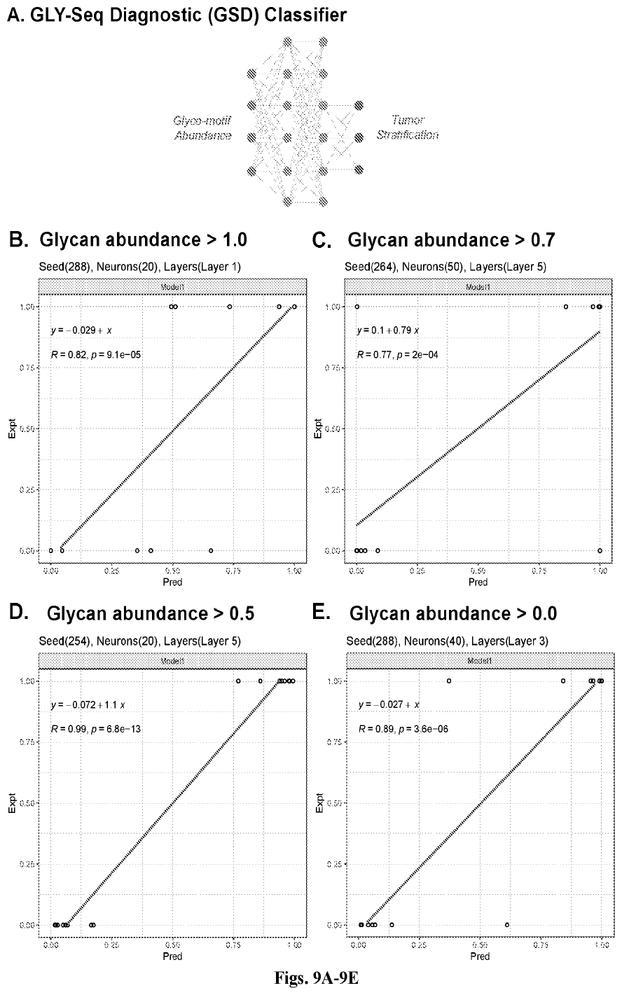

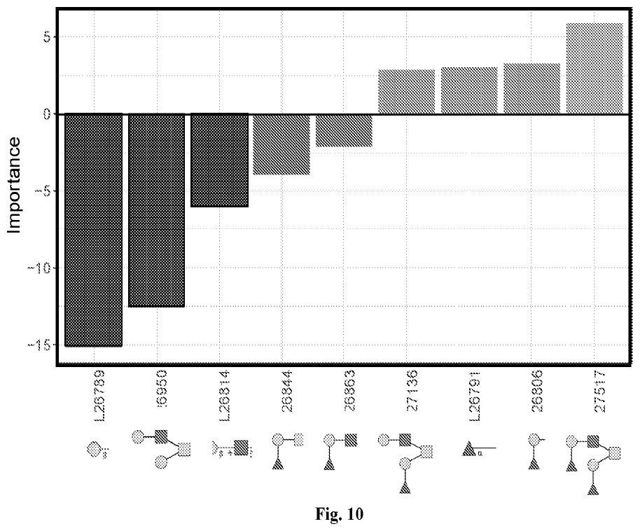

Lastly, we trained a neural network model on the GlyCompare derived glyco-motif profiles (see details in Methods; FIG. 9A). To determine the optimal neural network topology, we assessed the performance using different combinations of hidden layer size and neuron size in each layer. Based on the ten-fold cross-validation, our results show that the neural network with 1 hidden layer (20 neurons) has the best average prediction power, in which the best model has excellent performance (Pearson correlation coefficient (R)=0.82, p<9.1e−05) (FIG. 9B). Moreover, the model shows great stratification performance (R>0.77) for the glyco-motif profiles derived from the reconstructed glycoprofiles with different lowly expressed glycans filtered (FIGS. 9C-9E). To further understand the importance of input glyco-motifs in neural networks, the relative importance of each glyco-motif is quantified as the sum of the product of raw input-hidden and hidden-output connection weights between each input and output neuron and sums the product across all hidden neurons.^{26,27} Consistent with the differential analysis results (FIGS. 7A-7B and 8), our results show that three glyco-motifs: L26789, L26950 and L26814 are essential for accurately stratifying the gastric disease glycoprofiles (FIG. 10). These results suggest that GLY-Seq Diagnostics (GSD) classifier not just can successfully classify glyco-motif profile into different tumor class but also identify the critical glyco-motifs for the disease stratification.

### Example 2—Prostate Specific Antigen (PSA) Glycosylation for Diagnosis of Prostate Cancer

Reconstructing the Mass-Spectrometry-Based Glycoprofiles from Lectin Profiles with a Neural Network

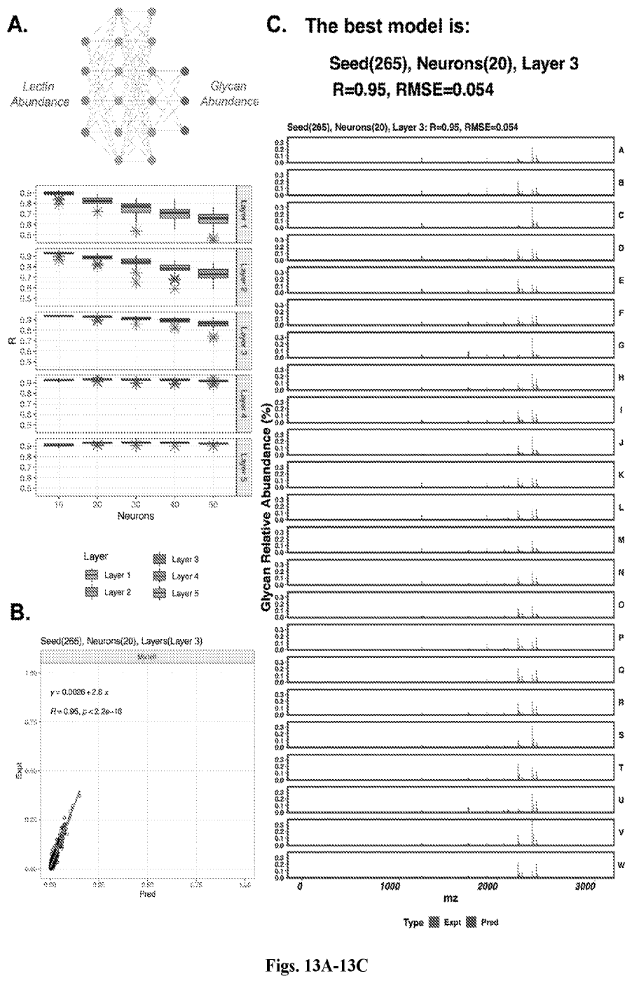

In this example, we trained a neural network model on the PSA glycomics data37 (see details in Methods and Table 6). To determine the optimal neural network topology, we assessed the performance using different combinations of hidden layer size and neuron size in each layer. Based on the ten-fold cross-validation, our results show that the neural network with 3 hidden layer (20 neurons) has the best average prediction power, in which the best model has excellent performance (Pearson correlation coefficient (R)=0.95, p<2.2e−16) (FIGS. 13A-13C). To further understand the importance of input lectins in neural networks, the relative importance of each lectin is quantified as the sum of the product of raw input-hidden and hidden-output connection weights between each input and output neuron and sums the product across all hidden neurons.^{26,27} Our results suggest that two lectins: TJA-I³⁸ that specifically bound to terminal sialic acids (Siaα(2-6)Gal) and GalNAc, and UDA³⁸ bound to terminal mannose Manα(1-6), type 2 LacNAc, and Chitin fragment antigens are essential for accurately reconstructing the PSA glycoprofiles (FIGS. 13A-13C). While these two lectins should be critically designated as probes to detect glycans in the GLY-Sequencing detection device (e.g., Microfluidic platforms, FACS, sequencing, etc.) for the prostate tumor glycans profiled here, trial runs on all lectins can be used to identify the most important lectins for profiling the glycan patterns in the other disease of interest.

TABLE 3
Sample information for PSA N-glycan profiling of prostate tumor

| | | Prostate | PSA serum | | | Clincial | |
|---|---|---|---|---|---|---|---|
| | Age | volume | concentration | | | Tumor | |
| Patient | group | (mL) | (ng/mL) | Gleason | Pca | staging | Disease |
| A | 1950-1959 | 50-75 | 2.5-5.0 | NA | Non-PCa | Normal | Normal |
| B | 1950-1959 | 75-100 | 2.5-5.0 | NA | Non-PCa | Normal | Normal |
| C | 1950-1959 | 25-50 | 5.0-7.5 | NA | Non-PCa | Normal | Normal |
| D | 1970-1979 | 50-75 | 5.0-7.5 | NA | Non-PCa | Normal | Normal |
| E | 1950-1960 | 50-75 | 5.0-7.5 | NA | Non-PCa | Normal | Normal |
| F | 1940-1949 | 25-50 | 2.5-5.0 | NA | Non-PCa | Normal | Normal |
| G | 1960-1969 | 50-75 | 7.5-10.0 | NA | Non-PCa | Normal | Normal |
| H | 1940-1949 | 25-50 | 15.0-100.0 | NA | Non-PCa | Normal | Normal |
| I | 1940-1949 | 50-75 | 10.0-15.0 | NA | Non-PCa | Normal | Normal |
| J | 1940-1949 | >100 | 5.0-7.5 | NA | Non-PCa | Normal | Normal |
| K | 1940-1949 | 25-50 | 2.5-5.0 | 3 + 4 | PCa | T1c | Tumor |
| L | 1930-1939 | 50-75 | 2.5-5.0 | 3 + 4 | PCa | T1c | Tumor |
| M | 1950-1959 | 50-75 | 10.0-15.0 | 3 + 3 | PCa | T2 | Tumor |
| N | 1940-1949 | 25-50 | 5.0-7.5 | 3 + 4 | PCa | T1c | Tumor |
| O | 1940-1949 | 50-75 | 2.5-5.0 | 4 + 3 | PCa | T1c | Tumor |
| P | 1950-1959 | 75-100 | 10.0-15.0 | 3 + 3 | PCa | T1c | Tumor |
| Q | 1950-1959 | 25-50 | 2.5-5.0 | 3 + 4 | PCa | T1c | Tumor |
| R | 1940-1949 | 50-75 | 7.5-10 | 3 + 3 | PCa | T1c | Tumor |
| S | 1940-1949 | 25-50 | 2.5-5.0 | 3 + 3 | PCa | T1c | Tumor |
| T | 1930-1939 | 50-75 | >100 | 4 + 5 | PCa | T2 | Tumor |
| U | 1940-1949 | 50-75 | 5.0-7.5 | 3 + 3 | PCa | T2 | Tumor |
| V | 1950-1959 | 50-75 | 7.5-10 | 3 + 3 | PCa | T1c | Tumor |
| W | 1940-1949 | >100 | 7.5-10.0 | 3 + 4 | PCa | T1c | Tumor |

### GlyCompare Reveals Tumor-Differential N-Glycans by Increasing the Statistical Power of Glycomics Data

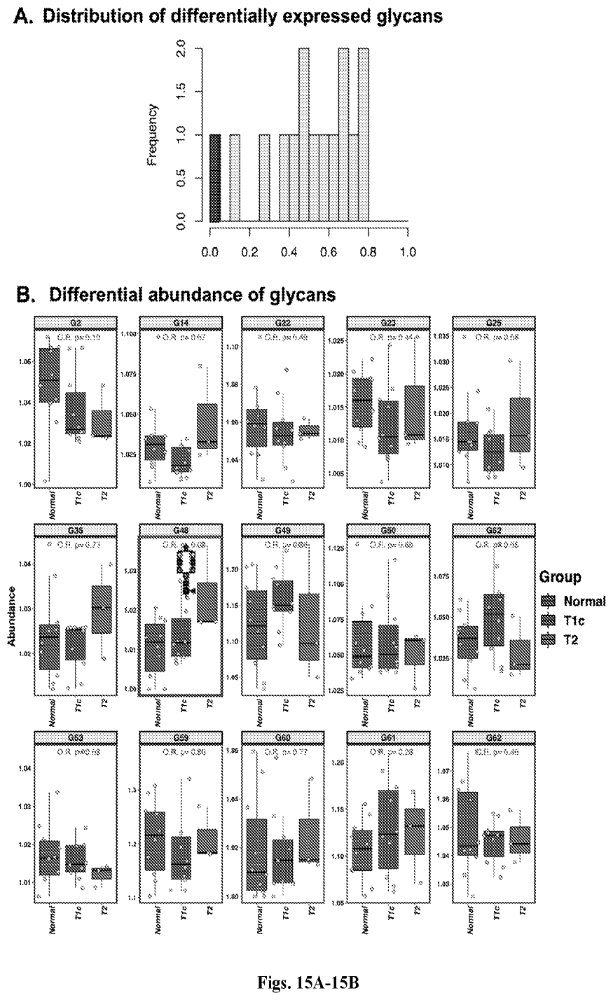

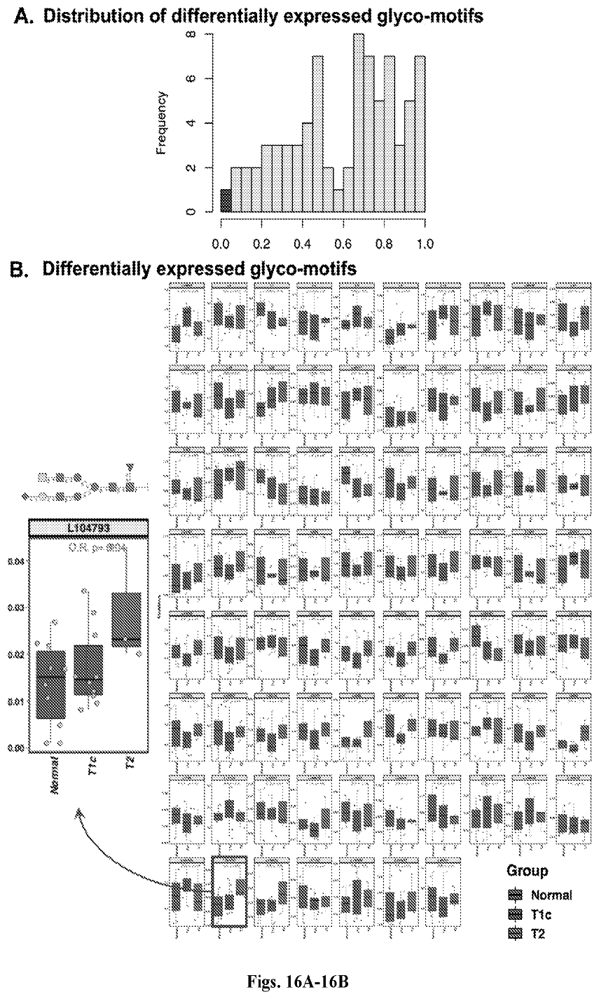

To investigate whether we can computationally identify glycan substructure signatures from glycomics data for stratifying subjects between normal and cancer, we started by using our method-GlyCompare (FIGS. 11A-11f) to calculate substructure abundance for experimentally measured PSA N-glycans³⁷. In the examination of the PSA N-glycans from tumor and normal samples, glycan abundance and motif abundance were compared (FIGS. 11A-11f, 15A-15B and 16A-16B). We found that one measured glycan structure significantly distinguished between T2 stage tumor, T1c stage tumor, and normal following ordinal regression test (P value=0.03, FIGS. 15A-15B). After substructure decomposition using GlyCompare, we also found only one significant (P value=0.04) PSA glycan motif increased in prostate cancer³⁷ (FIGS. 16A-16B). The observation of significantly distinct substructures suggests GlyCompare can detect the distinguishing condition-enriched structures, and further showed continuity across similar structures was not evident in the original study.³⁷

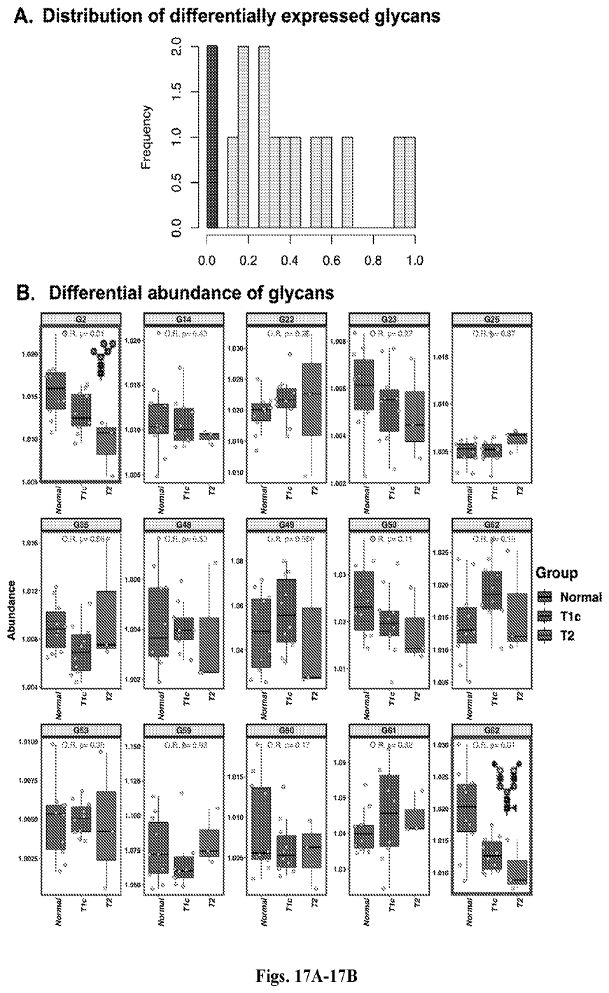

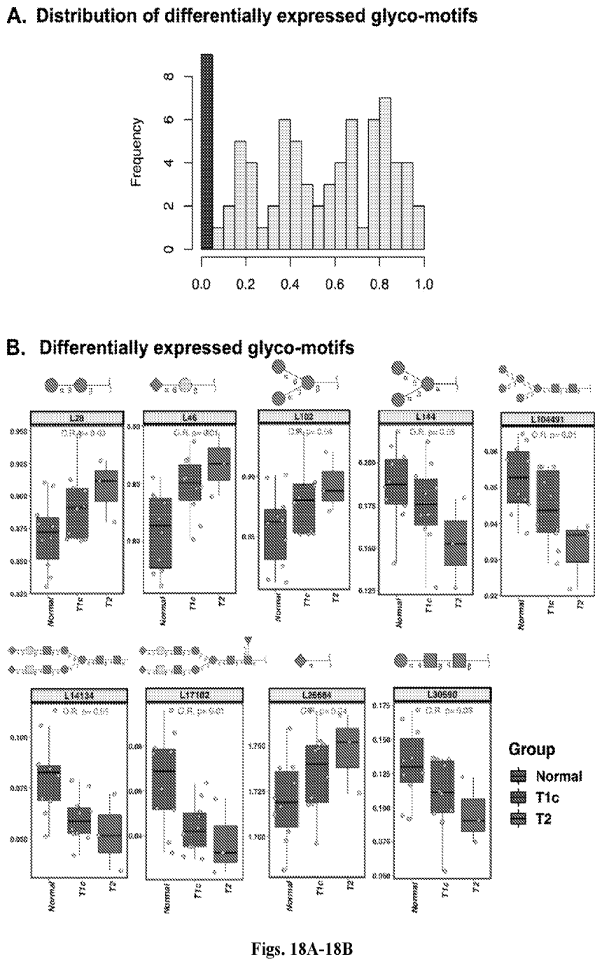

Next, we tested whether we could identify glycan substructure signatures from the reconstructed glycomics data (FIGS. 13A-13C) using the best neural network model to predict glycan structure from lectin binding patterns. In the re-examination of the PSA N-glycans from tumor and normal samples, we found two whole-glycan structures significantly distinguished between tumor and normal following ordinal regression test (p value<0.05; FIGS. 17A-17B). After substructure decomposition using GlyCompare, we found five significantly decreased (IDs=L144, L104491, L14134, L17102, and L30590; p value<0.05) and four significantly increased (IDs=L28, L46, L102, and L26664); p value<0.05) PSA glycan motifs in prostate cancer³⁷ (FIG. 18B). Through this we demonstrate the increase in statistical power when using substructures. All these results demonstrated that GlyCompare can process the reconstructed glycomics data to clarify analyses and greatly improve statistical power.

The GLY-Seq Diagnostics (GSD) Classifier Can Successfully Classify Glyco-Motif Profile into Different Clinical Tumor Stages Of Prostate Cancer

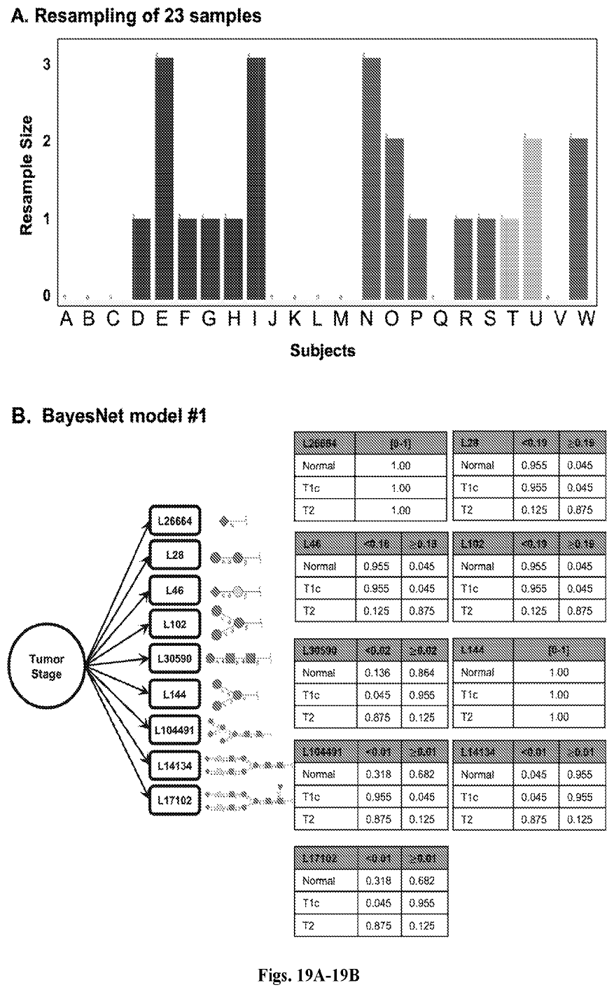

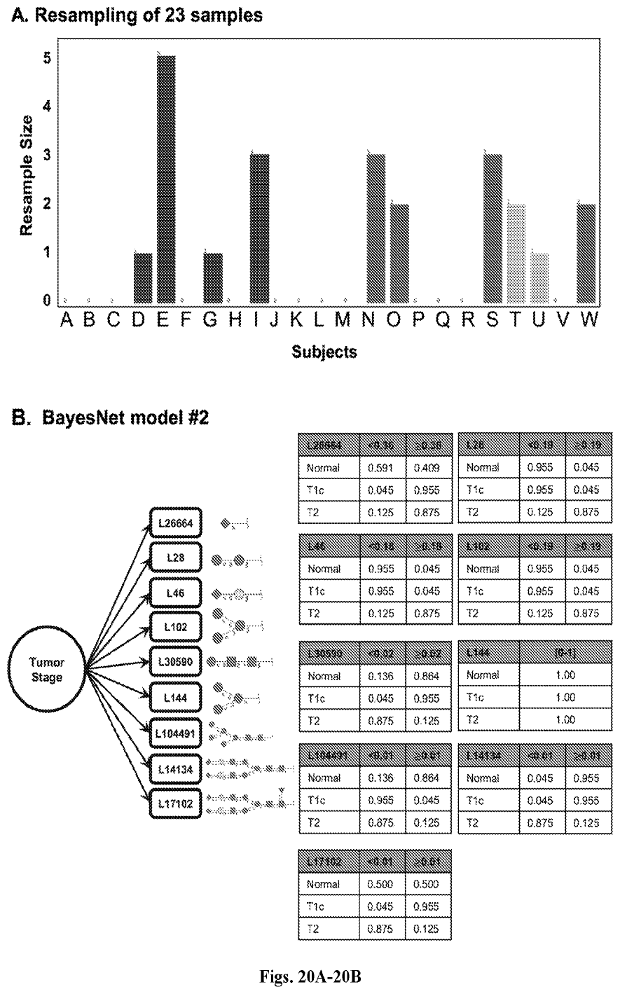

Lastly, we trained a Bayesian network model on the GlyCompare derived glyco-motif profiles (FIGS. 19A-19B and 20A-20B). Since the original dataset contains very imbalanced tumor state samples (10 normal, 10 T1c, and 3 T2), we thus resampled two datasets from original dataset with the same size (23 samples) for a fair evaluation. Based on the one-out cross-validation, our results show that the Bayesian Network model has great prediction power, in which both model-I (Table 4) and model-II (Table 5) has excellent performance (overall correctly classified rate=82.6% and 95.7%). Most important of all, the conditional probability (FIGS. 19B and 20B) quantified by the Bayesian Network can help us to identify the critical glyco-motifs for the disease stratification. These results therefore suggest that GLY-Seq Diagnostics (GSD) classifier can successfully classify glyco-motif profile into different tumor class and provide insightful information about the biomarkers for scientists to investigate their mechanistic mechanisms in the disease.

TABLE 4
Confusion Matrix for BayesNet Model-I

| | | Truth Stage Class | | | |
|---|---|---|---|---|---|
| | | Normal | T1c | T2 | Precision |
| Model | Normal | 7 | 1 | 0 | 87.50% |
| Prediction | T1c | 3 | 9 | 0 | 75% |
| | T2 | 0 | 0 | 3 | 100% |
| | Recall | 70% | 90% | 100% | |

TABLE 5
Confusion Matrix for BayesNet Model-II

| | | Truth Stage Class | | | |
|---|---|---|---|---|---|
| | | Normal | T1c | T2 | Precision |
| Model | Normal | 9 | 0 | 0 | 100% |
| Prediction | T1c | 0 | 10 | 0 | 100% |
| | T2 | 1 | 0 | 3 | 75% |
| | Recall | 90% | 100% | 10% | |

Mass-spectrometry-based cancer diagnostics are expensive and laborious. Recent advances in computational biology tools with lectin profiling technologies offers a novel opportunity to understand how aberrant variations in glycosylation lead to pathogenesis of human diseases. Here we showed the GlyCompare can be used to process glycomics data for enhanced diagnostic capabilities. We further developed a disease diagnostic system termed Gly-Seq Diagnostics (GSD) that can be deployed in the glycosequencing platform to enable high throughput, NGS-based diagnostics. The results warrant three major conclusions: (1) the proposed method can accurately reconstruct high-resolution glycome at both disease state and normal state that robustly tolerate the lowly expressed glycoform; (2) the reconstructed glycoprofiles can be transformed to glyco-motif profiles that increases the statistical power of glycomics data; and (3) the developed GSD-classifier is powerful for accurately stratifying the glyco-motif profiles into gastric tumor class. The successful development of GSD-system presents not only a unique solution to the challenge of tumor glycan diagnostics, but also demonstrates a novel strategy for investigating glycosylation pathogenic process of many other human diseases. The developed method can greatly facilitate investigating glycosylation in human diseases and translating the leveraged knowledge into clinical diagnostic, prognostic, and therapeutic applicability.

### Materials and Methods

### Simulated Lectin Profiles

Lectins have been known for their highly specific carbohydrate binding.^{30,31} To distinguish heterogeneity among the glycoprofiles, we selected a set of 14 lectins (Table 6) that can capture the entire glycome for O-linked protein glycosylation in a gastric tumor dataset²⁴. The selected lectins distinguish 14 specific glycan structural features of O-linked glycans.¹⁸ Given a glycoprofile, the lectin binding profile (LP) can be generated by using Equations 1: LP_{k,j}=GPg_{k,i}*LPg_{i,j}, where LP_{k,j} is the lectin binding profiles for given glycoprofiles, each row represents a specific glycoprofile k and each column represents a lectin j; GPg_{k,i} is the signal intensity (relative MS/HPLC intensity) of glycan i in the given glycoprofile k; and LPg_{i,j} is the lectin binding profiles for given glycan i and lectin j. Here, we applied this method to generate the seventeen lectin profiles from the experimentally measured glycoprofiles of gastric disease.²⁴ These simulated lectin profiles were used for further analysis in this study.

TABLE 6
Selected lectins for O-glycan lectin profiling

| Lectin_ID | Lectin_name | Glycan specificity* | Note |
|---|---|---|---|
| UEA-I | Ulex europaeus | Fuc(a1-2)Gal(b1-4)GlcNAc | H-type 2 |
| MAL_I | Maackia amurensis | Neu5Ac(a2-3)Gal(b1-4)GlcNAc | Sia |
| TJA-I | Trichosanthes | Neu5Ac(a2-6)GalNAc | Sia |
| | japonica | | |
| RCA120 | Ricinus communis | Gal(b1-4)GlcNAc | terminal Gal |
| BPL | Bauhinia purpurea | Gal(b1-3)GalNAc | Lewis x |
| TJA-II | Tanthes japonica | Fuc(a1-2)Gal | Fuca1-2Galb1 |
| EEL | Euonymus europaeus | Gal(a1-3)Gal(b1-4)GlcNAc | Galili_antigen |
| LEL | Lycopersicon | Gal(b1-4)GlcNAc(b1-3)Gal(b1- | PolyLacNAc |
| | esculentum | 4)GlcNAc | |
| Jacalin | Artocarpus integrifolia | GlcNAc(b1-3)GalNAc | Core3 |
| WFA | Wisteria floribunda | GalNAc(b1-4)GlcNAc | LacdiNAc |
| ACA | Amaranthus caudatus | Neu5Ac(a2-3)Gal(b1-3)GalNAc | sialyl T |
| HPA | snail, Helix pomatia | GalNAc(a1-3)GalNAc | Core5 |
| VVA | Vicia villosa | Gal(b1-3)GlcNAc | LacNAc_typel |
| MAH | Maackia amurensis | Neu5Ac(a2-3)Gal(b1-3)[Neu5Ac(a2- | disialyl-T |
| | | 6)]GalNAc | |

*The sugar abbreviations of ‘Fuc’, ‘Gal’, ‘GalNAc’, ‘Glc’, ‘GlcNAc’, and ‘Neu5Ac’ represent L-Fucose, D-Galactose, N-Acetylgalactosamine, D-Glucose, N-Acetylglucosamine, and Sialic Acid respectively.

### Development of the GLY-Seq Diagnostics (GSD) Approach

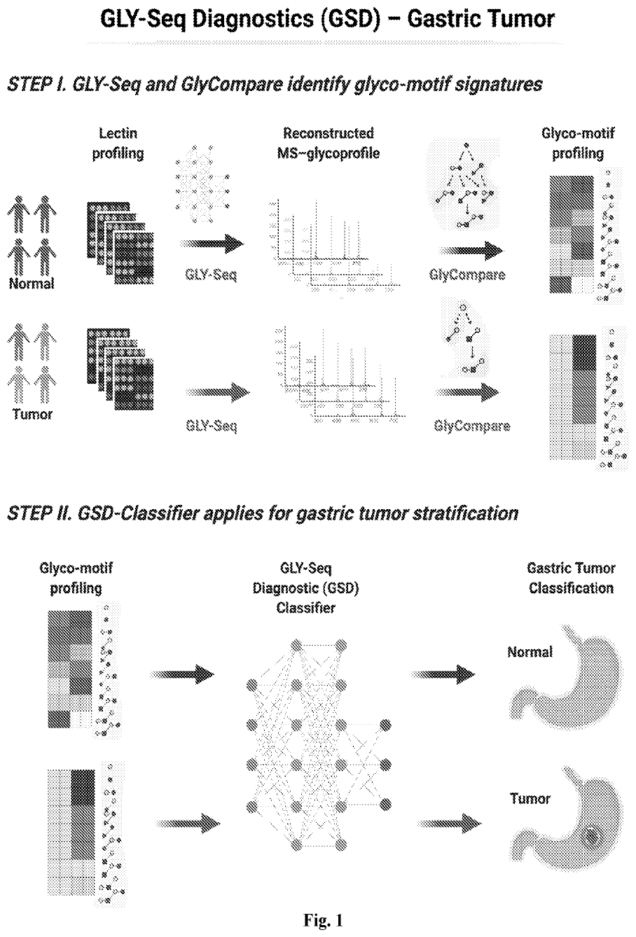

The presented GSD-system comprises of two steps (FIG. 1): (STEP I) Reconstructing glycoprofiles from lectin profiles and transform the glycoprofiles to derive glyco-motif profiles. A schematic view of samples (e.g., cells or tissues that present glycans), in which the glycans to be measured can be on tissues, single cells, protein samples (e.g., proteins captured on beads or a surface), lipid micelles, immobilized proteins, glycans or other molecules. Glycan motifs can be identified with carbohydrate-binding molecules, such as lectins, Lectenz, antibodies, nanobodies, aptamers, small molecules (e.g., boronic acids), etc. The resulting lectin binding patterns reveal the presence of key epitopes, and the original glycan species (MS-glycoprofile) is recapitulated from lectin binding patterns via a neural network. Lastly, by applying GlyCompare²¹, the glycan substructure (glyco-motif profile) of each glycan can be derived for each sample. In each sample, the interdependent relationship between the glycan species is considered, revealing important intermediary structures in a larger glycan synthesis network. This improves statistical power of analysis involving large glycomics datasets. (STEP II) GSD-classifier stratify the glyco-motif profile to tumor class. The glyco-motif profiles are subsequently used to train and interrogate an AI model that detects tumor-driven glycan signals and classified the samples into different tumor classes.

Applying GlyCompare for Computing Glycan Substructure Profiles from Glycoprofiles.

We summarize the procedures of GlyCompare²¹ that were used for generating the glyco-motif profiles from the studied glycoprofiles (FIGS. 11a-11f). First, glycoprofiles are parsed into glycans with abundance. In each glycoprofile, the glycans manually annotated with the GlycoCT format. GlycoCT formatted glycans are loaded into Python (version 3+) and initialized as glypy.glycan objects using the Glypy (version 0.12.1).³² Assuming we have a glycoprofile i, the corresponding abundance of each glycan j in glycoprofile i is represented by gᵢⱼ. Second, glycans are annotated with glycan substructure information, and this information is transformed into the substructure vector. Substructures within a glycan are exhaustively extracted by breaking down each linkage or a combination of linkages of the studied glycan. All substructures extracted are merged into a substructure set S. Substructures are sorted by the number of monosaccharides and duplicates are removed. Then, each glycan is matched to the substructure set S, producing a binary glycan substructure presence (1) or absence (0) vector, xᵢⱼ. Last, a substructure (abundance) vector is calculated as Pᵢ=xᵢⱼ*gᵢⱼ representing the abundance of the substructures s in this glycoprofile i. The glyco-motif profiles were derived by computing all the glycoprofiles.

### Substructure Decomposition of Mass-Spectrometry-Based Measurements of Mucin-Type O-Glycans to Clarify Tumor-Specific Glycan Epitopes

We analyzed structural mucin-type O-glycan abundance²⁴. Mucin-type O-glycans were originally measured by liquid chromatography and mass spectrometry (LC-MS), structures were manually annotated using empirical masses from Unicarb-DB³³. Pre-processing of these data was restricted to reformatting for input into Glycompare-compatible abundance matrix and structure annotation. Formatted data were normalized using probabilistic quotient normalization³⁴. Substructure abundances and motif extraction were performed using a monosaccharide core for thereby focusing analysis on epitope motifs.

Using the mucin-type O-glycan data, we examined both the original glycan abundance data and the motif-level abundance decomposition. Glycan and motif structure abundance was compared across cancer and non-cancer samples using two-sample t-tests; p-values were multiple-test corrected using false discovery rate³⁵.

In addition to being applicable to mass spectrometry data, GlyCompare can be used with compositional site-specific N-glycan abundance²⁵. As an example, we used site-specific N-glycan composition that was measured using activated-ion electron transfer dissociation (AI-ETD), the log of localized spectra count for each site-specific composition, and used this to represent abundance. Pre-processing of these data was restricted to reformatting for input into a GlyCompare-compatible abundance matrix and structure annotation. Formatted data were normalized using probabilistic quotient normalization³⁴. Substructure abundances and motif extraction were performed using compositional monosaccharides thereby focusing analysis on epitope motifs.

Examining site-specific N-glycan compositional data from mouse brain, we used a slightly modified method to compute compositional substructure abundance from compositional abundance. To calculate compositional substructure, we sum over larger and subsuming structures in a compositional network. Consider the compositional abundance of a structure: HexNac(p)Hex(q)Fuc(r). Instead of abundance of HexNAc=p, Hex=q, and Fuc=r, we examine the compositional abundance for all measurements where HexNAc>=p, Hex>=q, and Fuc>=r. The network structure can be constrained to provide additional insight (e.g., Glyconnect Compozitor³⁶), currently, the aggregation criteria remain simple. In analyzing these data, we explored trends in correlation between observed compositional vs compositional-substructure abundance.

GlyCompare can be Used for Analysis of Site-Specific Glycosylation Data from Glycoproteomics

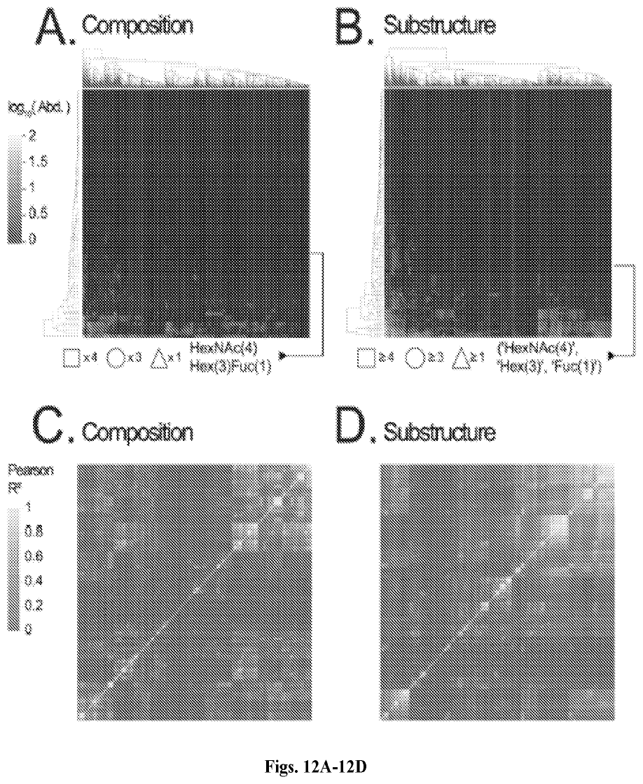

Examining site-specific N-glycan compositional data from mouse brain, we found that the decomposition of composition abundance into composition substructure abundance reveals additional potential signal. As previously shown, the sparsity of the abundance matrix decreases, and the comparability of profiles is improved when glycan data is aggregated over substructures (FIGS. 12A, 12B). Further, the correlation structure of substructure aggregated abundance (FIG. 12D) appears more robust than its compositional counterpart (FIG. 12C); there are more clusters with clearer borders, multiple clear off-diagonal clusters and the median R² is approximately doubled. While it is possible that the higher correlation is indicative of an increased background, that is unlikely considering the increase in visible correlation is structured, not randomly distributed through the background. These compositional data can be similarly used by processing with GlyCompare and analysis using machine learning and AI approaches as shown above for mass spectrometry data.

### References

1. Vargas, J. D. & Lima, J. A. C. Coronary artery disease: a gene-expression score to predict obstructive CAD. Nat. Rev. Cardiol. 10, 243-244 (2013).
2. van't Veer, L. J. et al. Gene expression profiling predicts clinical outcome of breast cancer. Nature 415, 530-536 (2002).
3. Blank, P. R. et al. Cost-effectiveness analysis of prognostic gene expression signature-based stratification of early breast cancer patients. Pharmacoeconomics 33, 179-190 (2015).
4. Myers, M. B. Targeted therapies with companion diagnostics in the management of breast cancer: current perspectives. Pharmgenomics. Pers. Med. 9, 7-16 (2016).
5. Manimala, J. C., Li, Z., Jain, A., VedBrat, S. & Gildersleeve, J. C. Carbohydrate array analysis of anti-Tn antibodies and lectins reveals unexpected specificities: implications for diagnostic and vaccine development. Chembiochem 6, 2229-2241 (2005).
6. Papamichael, M. M. et al. Application of metabolomics in pediatric asthma: Prediction, diagnosis and personalized treatment. Metabolites 11, 251 (2021).
7. Varki, A. Biological roles of glycans. Glycobiology 27, 3-49 (2017).
8. Haukedal, H. & Freude, K. K. Implications of glycosylation in Alzheimer's disease. Front. Neurosci. 14, 625348 (2020).
9. Lau, K. S. & Dennis, J. W. N-Glycans in cancer progression. Glycobiology 18, 750-760 (2008).
10. Reilly, C., Stewart, T. J., Renfrow, M. B. & Novak, J. Glycosylation in health and disease. Nat. Rev. Nephrol. 15, 346-366 (2019).
11. Pinho, S. S. & Reis, C. A. Glycosylation in cancer: mechanisms and clinical implications. Nat. Rev. Cancer 15, 540-555 (2015).
12. Syed, P. et al. Role of lectin microarrays in cancer diagnosis. Proteomics 16, 1257-1265 (2016).
13. Hart, G. W. & Copeland, R. J. Glycomics hits the big time. Cell 143, 672-676 (2010).
14. Cummings, R. D. & Pierce, J. M. The challenge and promise of glycomics. Chem. Biol. 21, 1-15 (2014).
15. Cummings, R. D. & Michael Pierce, J. Handbook of Glycomics. (Academic Press, 2009).
16. Gupta, G., Surolia, A. & Sampathkumar, S.-G. Lectin microarrays for glycomic analysis. OMICS 14, 419-436 (2010).
17. Satomaa, T. et al. Analysis of the human cancer glycome identifies a novel group of tumor-associated N-acetylglucosamine glycan antigens. Cancer Res. 69, 5811-5819 (2009).
18. Zhang, L., Luo, S. & Zhang, B. The use of lectin microarray for assessing glycosylation of therapeutic proteins. MAbs 8, 524-535 (2016).
19. Zhang, L., Luo, S. & Zhang, B. Glycan analysis of therapeutic glycoproteins. MAbs 8, 205-215 (2016).
20. National Research Council, Division on Earth and Life Studies, Board on Life Sciences, Board on Chemical Sciences and Technology & Committee on Assessing the Importance and Impact of Glycomics and Glycosciences. Transforming Glycoscience: A Roadmap for the Future. (National Academies Press, 2012).
21. Bao, B. et al. Correcting for sparsity and non-independence in glycomic data through a systems biology framework. bioRxiv 693507 (2019) doi:10.1101/693507.
22. Guo, Y. et al. Lectin microarray and mass spectrometric analysis of hepatitis C proteins reveals N-linked glycosylation. Medicine 97, e0208 (2018).
23. Lewis, N. E., Chiang, W. T., Liang, C. & Sorrentino, J. T. Method of Measuring Complex Carbohydrates at the Nano-Scale. WO2022/026944.
24. Jin, C. et al. Structural diversity of human gastric mucin glycans. Mol. Cell. Proteomics 16, 743-758 (2017).
25. Riley, N. M., Hebert, A. S., Westphall, M. S. & Coon, J. J. Capturing site-specific heterogeneity with large-scale N-glycoproteome analysis. Nat. Commun. 10, 1311 (2019).
26. Olden, J. An accurate comparison of methods for quantifying variable importance in artificial neural networks using simulated data. Ecological Modelling (2004) doi:10.1016/s0304-3800(04)00156-5.
27. Olden, J. D. & Jackson, D. A. Illuminating the “black box”: a randomization approach for understanding variable contributions in artificial neural networks. Ecological Modelling vol. 154 135-150 (2002).
28. Sibille, E. et al. Ganglioside profiling of the human retina: Comparison with other ocular structures, brain and plasma reveals tissue specificities. PLoS One 11, e0168794 (2016).
29. Koda, Y., Soejima, M., Liu, Y. & Kimura, H. Molecular basis for secretor type alpha(1,2)-fucosyltransferase gene deficiency in a Japanese population: a fusion gene generated by unequal crossover responsible for the enzyme deficiency. Am. J. Hum. Genet. 59, 343-350 (1996).
30. Hsu, K.-L., Pilobello, K. T. & Mahal, L. K. Analyzing the dynamic bacterial glycome with a lectin microarray approach. Nat. Chem. Biol. 2, 153-157 (2006).
31. Zielinska, D. F., Gnad, F., Wiśniewski, J. R. & Mann, M. Precision mapping of an in vivo N-glycoproteome reveals rigid topological and sequence constraints. Cell 141, 897-907 (2010).
32. Klein, J. & Zaia, J. glypy: An Open Source Glycoinformatics Library. J. Proteome Res. 18, 3532-3537 (2019).
33. Campbell, M. P. et al. Validation of the curation pipeline of UniCarb-DB: building a global glycan reference MS/MS repository. Biochim. Biophys. Acta 1844, 108-116 (2014).
34. Benedetti, E. et al. Systematic evaluation of normalization methods for glycomics data based on performance of network inference. Metabolites 10, 271 (2020).
35. Benjamini, Y. & Hochberg, Y. Controlling the false discovery rate: A practical and powerful approach to multiple testing. J. R. Stat. Soc. 57, 289-300 (1995).
36. Robin, T., Mariethoz, J. & Lisacek, F. Examining and fine-tuning the selection of glycan compositions with GlyConnect Compozitor. Mol. Cell. Proteomics 19, 1602-1618 (2020).
37. Kammeijer, G. S. M., Nouta, J., de la Rosette, J. J. M. C. H., de Reijke, T. M., Wuhrer, M. An In-Depth Glycosylation Assay for Urinary Prostate-Specific Antigen. Anal Chem. 90(7):4414-4421 (2018).
38. Bojar, D., Meche, L., Meng, G., Eng, W., Smith, D. F., Cummings, R. D., Mahal, L. K. A Useful Guide to Lectin Binding: Machine-Learning Directed Annotation of 57 Unique Lectin Specificities. ACS Chem Biol. (2022). doi:10.1021/acschembio.1c00689.
39. Bao, B. et al. Correcting for sparsity and interindependence in glycoms by accounting for glycan biosynthesis. Nat. Comm. 12:4988 (2021). doi:10.1038/s41467-021-25183-5.

What is claimed is:

1. A method of diagnosis, prognosis, or treatment of a subject for a disease or condition, the method comprising,
providing a sample comprising glycans or glycosylated molecules from the subject;
quantifying glyco-motif profiles in the sample, wherein substructures of glycans are used as features for classification; and
translating the quantified glyco-motif profile for diagnosis, prognosis, or treatment.

2. The method of claim 1, wherein the glyco-motif profiles are quantified by mass spectrometry, chromatography or by incubating the sample with more than one carbohydrate-binding molecule, and generating a carbohydrate-binding molecule profile and glycan structures are decomposed to their substructures and glyco-motifs.

3. The method of claim 2, wherein the carbohydrate-binding molecule profile is used to determine glycan structure using a machine learning approach trained from known glycoprofiles that map the carbohydrate-binding molecule profile to an actual glycoprofile.

4. The method of claim 3, wherein the glyco-motif profiles of each glycan are quantified to reveal biosynthetic intermediate abundance from measured glycans.

5. The method of claim 4, wherein the translating step is performed by employing a classifier trained from a known control based on glyco-motif profiles for diagnosis, prognosis, or treatment.

6. The method of claim 5, wherein substructures of glycans are used as features for classification using a method of machine learning, support vector machines, regression model, and/or neural networks.

7. The method of claim 1, wherein glyco-motif profiles are quantified by decomposing glycan measurements from mass spectrometry or chromatography.

8. The method of claim 1, wherein the disease or condition is cancer, metabolic disease, immune disease, inflammatory condition, congenital disorders of glycosylation, or reproductive health condition.

9. The method of claim 1, wherein the disease is gastric cancer or prostate cancer.

10. A computer system, comprising:
one or more processors; and
memory storing executable instructions that, as a result of execution, cause the one or more processors of the computer system to:
a. quantify sample glycoprofiles data derived by mass spectrometry or chromatography, or by binding of carbohydrate-binding molecules to glycans in the sample;
b. transform the quantified glycoprofiles to glyco-motif profiles based on previous training data between other related samples;
c. classify the glyco-motif profiles into a disease class, wherein substructures of glycans are used as features for classification; and
d. predict most likely disease classes based on previous training data between other related samples.

11. The system of claim 10, wherein the sample is a tissue, a cell, a biomolecule, or an oligosaccharide.

12. The system of claim 10, wherein the glycoprofiles are quantified by carbohydrate-binding molecules, and transformed based on previous training data between other samples, before translating to glyco-motif profiles.

13. The system of claim 12, wherein the carbohydrate-binding molecules are selected from a lectin, an antibody, a nanobody, an aptamer, or an enzyme.

14. The system of claim 10, wherein the quantifying is conducted by fluorescence microscopy, immunohistochemistry, biotin-streptavidin, or sequencing nucleotide barcodes.

15. The system of claim 10, wherein the classifying is conducted using trained algorithm approaches from convex optimization and/or machine learning, trained from known glycoprofiles.

16. The system of claim 10, wherein quantifying measurements are made at a single cell level or products form a single cell, and wherein the cells are assayed on a microfluidics chip or droplets.

17. The system of claim 10, wherein the transforming is conducted using a trained algorithm to generate glyco-substructures.

18. The system of claim 10, wherein the predicting is conducted using trained algorithm approaches from machine learning, support vector machine, regression model and/or neural networks trained from known glycoprofiles.

19. The system of claim 10, wherein the disease or condition is cancer, metabolic disease, immune disease, inflammatory condition, congenital disorders of glycosylation, or reproductive health condition.

20. The system of claim 10, wherein the disease is gastric cancer or prostate cancer.
